(19) United States
(12) Patent Application Publication
(10) Pub. No.: US 2026/0151110 A1
(43) Pub. Date: Jun. 4, 2026

(54) ULTRASOUND IMAGING SYSTEM AND METHOD FOR CONTROLLING ULTRASOUND IMAGING SYSTEM

(71) Applicant: SAMSUNG MEDISON CO., LTD., Hongcheon (KR)

(72) Inventors: Yoonjae LEE, Hongcheon (KR); Jaehyuk LEE, Hongcheon (KR); Chulhee YUN, Hongcheon (KR); Minjung SONG, Hongcheon (KR); Yuri SON, Hongcheon (KR); Ilgu KWON, Hongcheon (KR); Donggeon KONG, Hongcheon (KR)

(21) Appl. No.: 19/373,007

(22) Filed: Oct. 29, 2025

(30) Foreign Application Priority Data
Nov. 29, 2024 (KR) 10-2024-0175902
Mar. 19, 2025 (KR) 10-2025-0035556
Sep. 5, 2025 (KR) 10-2025-0126939

(51) Int. Cl. A61B 8/00

(52) U.S. Cl. CPC A61B 8/485; A61B 8/463; A61B 8/5207; A61B 8/5223; A61B 8/5292

(57) ABSTRACT

A control method of an ultrasound imaging system comprises: obtaining a cine image, which comprises a plurality of image frames, by aligning an elasticity image generated based on a shear wave tracking signal received from a probe on the same time stamp and an elasticity reliability image generated based on parameters obtained from the shear wave tracking signal from the probe, with an ultrasound image generated based on an echo signal received from the probe; determining an elastic modulus measurement suitability of each of the plurality of image frames based on elasticity image data and elasticity reliability image data of each of the plurality of image frames; automatically extracting a plurality of image frames for measuring an elastic modulus based on the elastic modulus measurement suitability; automatically determining a position of measurement region of interest (ROI) of each of the extracted image frames based on the elasticity image data and the elasticity reliability image data of each of the extracted plurality of image frames; and displaying one of the extracted plurality of image frames and simultaneously displaying at least one of a first indicator for indicating an elastic modulus measurement suitability of the displayed image frame, a second indicator for indicating an automatically determined position of measurement ROI of the displayed image frame, and a third indicator for indicating a sort order of the displayed image frame among the extracted plurality of image frames.

### TECHNICAL FIELD

The present disclosure relates to an ultrasound imaging system and a control method thereof, and more particularly, to an ultrasound imaging system configured to improve measurement reliability and diagnostic efficiency by calculating and evaluating an elastic modulus measurement suitability for a plurality of image frames and by automatically extracting and displaying suitable image frames and measurement region of interest (ROI), and a control method thereof.

### BACKGROUND ART

In recent years, various medical imaging devices have been widely used in the medical field to obtain visual information about human tissues for the early diagnosis of various diseases or for surgical procedures. Representative examples of these medical imaging devices include ultrasound imaging devices, computed tomography (CT) devices, and magnetic resonance imaging (MRI) devices.

An ultrasound imaging device is a device that emits an ultrasound signal generated from a transducer of a probe to an object and receives information on the ultrasound signal reflected from the object so as to noninvasively obtain at least one image of a part inside the object (for example, soft tissue or blood flow). The ultrasound imaging device may be used for medical purposes such as observing the inside of the object, detecting foreign substances, and measuring injuries. The ultrasound imaging device has the advantages of higher stability than imaging devices that use X-rays, real-time display of images, and safety due to the absence of radiation exposure. Therefore, the ultrasound imaging device is widely used along with other imaging devices.

The ultrasound imaging device is widely used to noninvasively examine tissue structures within the human body or objects. Particularly, shear wave elastography is useful for quantitatively assessing an elastic modulus of the tissue. However, in clinical practice, the reliability of measurements may be reduced due to various factors, including respiration, heart rate, body shape, tissue inhomogeneity, and probe compression conditions.

In the conventional technology, image frames and measurement region of interests (ROIs) are often selected based on examiner's experience and subjective judgment, and thus there is limitation in measurement reproducibility and diagnostic standardization. Furthermore, the lack of sufficient technology for efficiently comparing, evaluating, and simultaneously displaying multiple image indices (for example, elasticity images, and reliability images) makes it difficult for examiners to intuitively assess the measurement quality.

### DISCLOSURE

### Technical Problem

The present disclosure is directed to providing a system capable of consistently evaluating measurement quality without subjective judgment of an examiner by calculating and scoring an elastic modulus measurement suitability of each image frame based on elasticity image and elasticity reliability image data.

Further, the present disclosure is directed to providing a system capable of automatically determining an optimal position of measurement region of interest (ROI) by comprehensively analyzing elasticity uniformity and reliability in extracted image frames, and capable of displaying a plurality of ROIs without mutual interference when the plurality of ROIs is required.

Further, the present disclosure is directed to providing a system capable of allowing an examiner to intuitively recognize quality and position information in real time by displaying a plurality of indicators simultaneously with an image frame and providing user customization and interaction functions.

### Technical Solution

One aspect of the present disclosure provides a control method of an ultrasound imaging system including: obtaining a cine image, which includes a plurality of image frames, by aligning an elasticity image generated based on a shear wave tracking signal received from a probe on the same time stamp and an elasticity reliability image generated based on parameters obtained from the shear wave tracking signal from the probe, with an ultrasound image generated based on an echo signal received from the probe; determining an elastic modulus measurement suitability of each of the plurality of image frames based on elasticity image data and elasticity reliability image data of each of the plurality of image frames; automatically extracting a plurality of image frames for measuring an elastic modulus based on the elastic modulus measurement suitability; automatically determining a position of measurement region of interest (ROI) of each of the extracted image frames based on the elasticity image data and the elasticity reliability image data of each of the extracted plurality of image frames; and displaying one of the extracted plurality of image frames and simultaneously displaying at least one of a first indicator for indicating an elastic modulus measurement suitability of the displayed image frame, a second indicator for indicating an automatically determined position of measurement ROI of the displayed image frame, and a third indicator for indicating a sort order of the displayed image frame among the extracted plurality of image frames.

The determining of the elastic modulus measurement suitability may include calculating elasticity uniformity standard deviation for each unit region from the elasticity image data of each of the plurality of image frames, and generating an elasticity uniformity STD map based on the elasticity uniformity standard deviation for each unit region; generating an average elasticity reliability map for each unit region from the elasticity reliability image data of each of the plurality of image frames; generating a first combined map for each of the plurality of image frames by synthesizing the elasticity uniformity STD map with the average elasticity reliability map; and determining the elastic modulus measurement suitability of each of the plurality of image frames by calculating an average value of the first combined map.

Additionally, an elasticity average value may be calculated from the elasticity image data and divided into the STD map to generate a representative elasticity uniformity STD map, and the representative elasticity uniformity STD map may be synthesized with the average elasticity reliability map to generate a first combined map of each of the plurality of image frames.

The displaying of one of the extracted plurality of image frames and simultaneously displaying the first indicator may include determining a color of a first display element included in the first indicator based on a preset score range to which the elastic modulus measurement suitability of the displayed image frame belongs.

The displaying of one of the extracted plurality of image frames and simultaneously displaying the first indicator may include determining the number of first display elements to allow the number of first display elements included in the first indicator to increase as the elastic modulus measurement suitability of the displayed image frame increases within the same score range.

The displaying of one of the extracted plurality of image frames may include simultaneously displaying a first ultrasound image in which the elasticity image is overlaid on the ultrasound image, and a second ultrasound image in which the elasticity reliability image is overlaid on the ultrasound image.

The displaying of one of the extracted plurality of image frames and simultaneously displaying the second indicator may include simultaneously displaying a second display element for indicating the position of the measurement ROI automatically determined on the first ultrasound image, and a third display element for indicating the position of the measurement ROI automatically determined on the second ultrasound image.

The automatically determining of the position of the measurement ROI of each of the extracted image frames may include generating an elasticity uniformity map by calculating an elasticity uniformity for each unit region from the elasticity image data of each of the extracted image frames; generating an average elasticity reliability map for each unit region from the elasticity reliability image data of each of the extracted image frames; calculating a composite score for each unit region by applying a first weighted value to the elasticity uniformity map and by applying a second weighted value to the average elasticity reliability, map and generating a second combined map on which the composite score for each unit region is reflected; extracting a unit region based on the composite score from the second combined map; and determining a position of the measurement ROI to allow the measurement ROI to include the extracted unit region.

The generating of the second combined map may include determining the first and second weighted values based on a user identification number obtained through a user interface or a communication interface.

The extracting of the unit region based on the composite score from the second combined map may include extracting one unit region in which the composite score is a maximum value. The determining of the position of the measurement ROI to allow the measurement ROI to include the extracted unit region may include determining a position of the measurement ROI so as to include one unit region in which the composite score is a maximum value.

The extracting of the unit region based on the composite score from the second combined map may include: additionally extracting at least one unit region in which the average elasticity reliability for each unit region is greater than or equal to a preset threshold value. The determining of the position of the measurement ROI to allow the measurement ROI to include the extracted unit region may include additionally determining at least one position of the measurement ROI so as to include at least one unit region in which the average elasticity reliability for each unit region is greater than or equal to the preset threshold value.

The displaying of one of the extracted plurality of image frames and simultaneously displaying the second indicator may include displaying, based on the position of the measurement ROIs being automatically determined as a plurality of positions, each position with a plurality of second indicators of different colors.

The displaying of one of the extracted plurality of image frames and simultaneously displaying the third indicator may include displaying a fourth display element provided to display information about a sort criterion; and displaying a fifth display element for displaying information about the sort order of the displayed image frames in response to the extracted plurality of image frames being sorted by the sort criterion.

The sort criterion may correspond to any one of sort in order of time or sort in order of elastic modulus measurement suitability. The control method of the ultrasound imaging system may include determining the sort criterion based on user input.

The displaying of the fifth display element may include overlaying and displaying a plurality of display units corresponding to each of the plurality of image frames extracted according to the sort criterion at equal intervals on a sort order display element; and highlighting and displaying a display unit corresponding to the displayed image frame.

The displaying of the fifth display element may include displaying each of the plurality of display units corresponding to each of the extracted plurality of image frames in a color determined based on a preset score range to which the elastic modulus measurement suitability of the corresponding extracted image frame belongs.

The automatically extracting of the plurality of image frames for measuring the elastic modulus based on the elastic modulus measurement suitability may include automatically extracting the image frame as an image frame for measuring the elastic modulus based on the elastic modulus measurement suitability of the image frame being greater than or equal to a reference value; and displaying, through a user interface, that none of the plurality of image frames has been extracted for measuring the elastic modulus based on the elastic modulus measurement suitability of all of the plurality of image frames included in the cine image being less than the reference value.

The automatically extracting of the plurality of image frames for measuring the elastic modulus based on the elastic modulus measurement suitability may include providing a guide for inducing resetting of the elastic modulus measurement suitability threshold value through the user interface based on the elastic modulus measurement suitability of all of the plurality of image frames included in the cine image being less than the reference value.

Another aspect of the present disclosure provides an ultrasound imaging system including: a probe configured to receive an echo signal and a shear wave tracking signal; an image processor configured to generate an ultrasound image based the echo signal, configured to generate an elasticity image based on the shear wave tracking signal, configured to generate an elasticity reliability image based on parameters obtained from the shear wave tracking signal, and configured to generate a cine image, which includes a plurality of image frames, by aligning the ultrasound image, the elasticity image and the elasticity reliability image; a display displaying the plurality of image frames; and a processor configured to determine an elastic modulus measurement suitability of each of the plurality of image frames based on elasticity image data and elasticity reliability image data of each of the plurality of image frames; configured to automatically extract a plurality of image frames for measuring an elastic modulus based on the elastic modulus measurement suitability; configured to automatically determine a position of measurement region of interest (ROI) of each of the extracted image frames based on the elasticity image data and the elasticity reliability image data of each of the extracted plurality of image frames; and configured to control the display to display one of the extracted plurality of image frames and simultaneously display at least one of a first indicator for indicating an elastic modulus measurement suitability of the displayed image frame, a second indicator for indicating an automatically determined position of measurement ROI of the displayed image frame, and a third indicator for indicating a sort order of the displayed image frame among the extracted plurality of image frames.

The processor may be configured to calculate elasticity uniformity standard deviation for each unit region from the elasticity image data of each of the plurality of image frames, and generate an elasticity uniformity STD map based on the elasticity uniformity standard deviation for each unit region; configured to generate an average elasticity reliability map for each unit region from the elasticity reliability image data of each of the plurality of image frames; configured to generate a first combined map for each of the plurality of image frames by synthesizing the elasticity uniformity STD map with the average elasticity reliability map; and configured to determine the elastic modulus measurement suitability of each of the plurality of image frames by calculating an average value of the first combined map.

Additionally, an elasticity average value may be calculated from the elasticity image data and divided into the STD map to generate a representative elasticity uniformity STD map, and the representative elasticity uniformity STD map may be synthesized with the average elasticity reliability map to generate a first combined map of each of the plurality of image frames.

The processor may be configured to determine a color of a first display element included in the first indicator based on a preset score range to which the elastic modulus measurement suitability of the displayed image frame belongs.

The processor may be configured to determine the number of first display elements to allow the number of first display elements included in the first indicator to increase as the elastic modulus measurement suitability of the displayed image frame increases within the same score range.

### Advantageous Effects

A process of calculating suitability and extracting region of interest (ROI) may be automated, and thus it is possible to perform measurement with consistent quality regardless of skill of an examiner, and to significantly improve standardization and reproducibility of diagnostic results.

Further, only high-quality frames may be automatically selected and low-reliability regions may be immediately identified, thereby reducing unnecessary re-measurement and shortening an examination time, and increasing diagnostic accuracy by reducing false positives and false negatives.

Further, indicator-based visualization and customization functions may allow a user to intuitively check the anatomical structure, elasticity properties, and reliability information of a target region for diagnosis on a single screen, thereby simplifying clinical workflow and improving decision-making speed.

### DESCRIPTION OF DRAWINGS

The present disclosure may be readily understood by the combination of the following detailed description and the accompanying drawings, wherein reference numerals refer to structural elements.

FIGS. 1A and 1B are block diagrams illustrating a configuration of an ultrasound imaging system according to one embodiment of the present disclosure.

FIGS. 2A, 2B, 2C, and 2D are views illustrating the ultrasound imaging system according to one embodiment of the present disclosure.

FIG. 3 is a view illustrating a configuration of an image frame displayed on a display device of the ultrasound imaging system according to one embodiment.

FIG. 4 is a flowchart illustrating a control method of the ultrasound imaging system according to one embodiment.

FIG. 5 is a flowchart illustrating a method for calculating an elastic modulus measurement suitability for each image frame according to one embodiment.

FIG. 6 is a block diagram briefly illustrating a configuration and processing of data input and output in a process of determining the elastic modulus measurement suitability.

FIG. 7 is a flowchart illustrating a method for automatically determining a measurement region of interest (ROI) position for each extracted image frame according to one embodiment.

FIG. 8 is a block diagram briefly illustrating a configuration and processing of data that is input and output in the automatic determination of measurement ROI position.

FIG. 9 is a view illustrating various indicators provided together with an image frame displayed on a display of the ultrasound imaging system according to one embodiment.

FIG. 10 is a view illustrating a configuration and display method of a first indicator according to one embodiment.

FIG. 11 is a view illustrating a configuration and display method of a second indicator according to one embodiment.

FIG. 12 is a view illustrating a configuration and display method of a plurality of second indicators according to another embodiment.

FIG. 13 is a view illustrating a configuration of a third indicator and a display method when sorted in order of time according to one embodiment.

FIG. 14 is a view illustrating a configuration of a third indicator and a display method when sorted in order of elastic modulus measurement suitability according to another embodiment.

FIG. 15 is a reference view illustrating color coding scheme of a fifth display element according to one embodiment.

FIG. 16 is a view illustrating various implementation methods of the fifth display element when sorted in order of time according to one embodiment.

FIG. 17 is a view illustrating various implementation methods of the fifth display element when sorted in order of measurement suitability according to one embodiment.

### MODES OF THE INVENTION

The description discloses the principles of the disclosure and discloses embodiments of the disclosure so that those skilled in the art will be able to practice the disclosure while clarifying the scope of the disclosure. The disclosed embodiments may be implemented in various forms.

Like reference numerals refer to like elements throughout the description. Well-known functions or constructions are not described in detail since they would obscure the one or more exemplar embodiments with unnecessary detail. Terms such as “module” or “unit” may be implemented by one or a combination of two or more of software, hardware, or firmware. According to embodiments, a plurality of “module” or “unit” may be implemented as a single element or a single “module” or “unit” may include a plurality of elements.

A singular expression corresponding to an item may include one item or a plurality of items unless otherwise indicated herein or clearly contradicted by context.

The expressions “A or B,” “at least one of A or/and B,” or “one or more of A or/and B,” A, B or C,” “at least one of A, B or/and C,” or “one or more of A, B or/and C,” and the like used herein may include any and all combinations of one or more of the associated listed items.

The term of “and/or” includes a plurality of combinations of relevant items or any one item among a plurality of relevant items.

The expressions “A or B,” “at least one of A or/and B,” or “one or more of A or/and B,” and the like used herein may include any and all combinations of one or more of the associated listed items. For example, the term “A or B,” “at least one of A and B,” or “at least one of A or B” may refer to all of the case (1) where at least one A is included, the case (2) where at least one B is included, or the case (3) where both of at least one A and at least one B are included.

Herein, the expressions “a first”, “a second”, “the first”, “the second”, etc., may simply be used to distinguish an element from other elements, but is not limited to another aspect (importance or order) of elements.

In addition, terms such as “front surface”, “rear surface”, “upper surface”, “lower surface”, “side surface”, “left side”, “right side”, “upper portion”, and “lower portion” used in the present disclosure are defined based on the drawings, and the shape and position of each component are not limited by these terms.

In this disclosure, the terms “including”, “having”, and the like are used to specify features, numbers, steps, operations, elements, components, or combinations thereof, but do not preclude the presence or addition of one or more of the features, elements, steps, operations, elements, components, or combinations thereof.

When an element is said to be “connected”, “coupled”, “supported” or “contacted” with another element, this includes not only when elements are directly connected, coupled, supported or contacted, but also when elements are indirectly connected, coupled, supported or contacted through a third element.

Throughout the description, when an element is “on” another element, this includes not only when the element is in contact with the other element, but also when there is another element between the two elements.

Hereinafter an ultrasonic device according to various embodiments will be particularly described with reference to the attached drawings. In the description with reference to the attached drawings, identical or corresponding components are assigned similar reference numerals, and redundant descriptions thereof may be omitted.

In the present disclosure, an ‘object’ refers to a subject to be imaged, and may include a human, an animal, or a part thereof. For example, the object may include a part of the body (such as an organ or system) or a phantom.

In the present disclosure, the term ‘ultrasound image’ may mean an image of an object generated or processed based on an ultrasonic signal (echo signal) transmitted to the object and reflected from the object.

In the present disclosure, the expression ‘robot arm supports A’ may mean that a robot arm grasps A, is docked to A, and/or fixes A, and may include that a robot arm directly or indirectly supports A.

Hereinafter exemplary embodiments of the present disclosure will be described in detail with reference to the accompanying drawings.

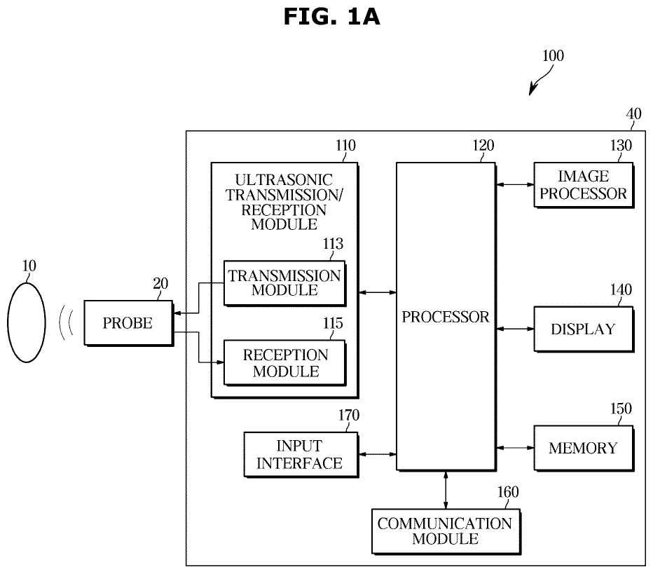

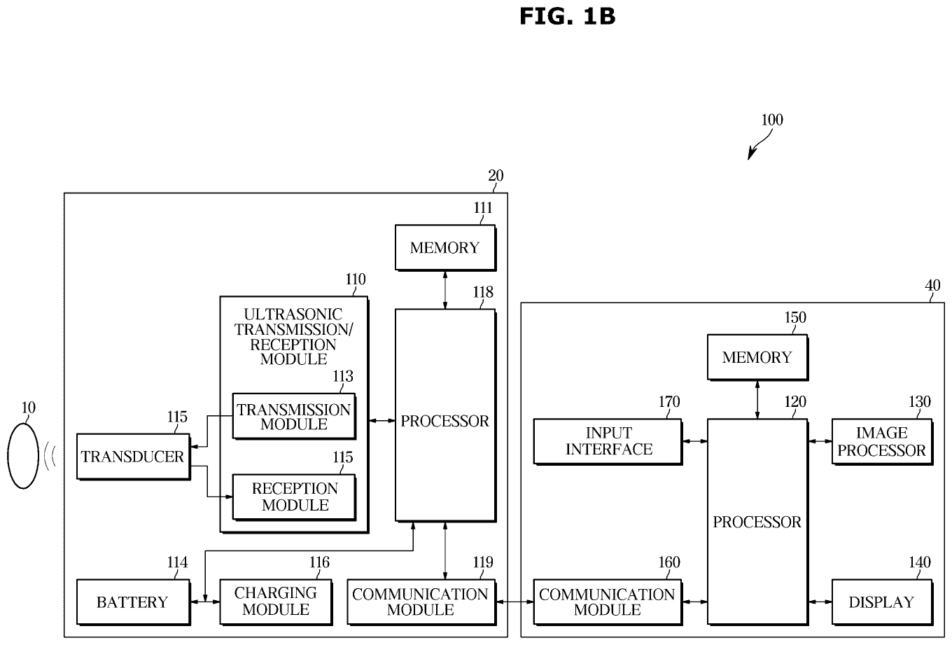

FIGS. 1A and 1B are block diagrams illustrating a configuration of an ultrasound imaging system according to one embodiment of the present disclosure.

Referring to FIGS. 1A and 1B, an ultrasound imaging system 100 may include a probe 20 and an ultrasound imaging device 40.

The ultrasound imaging device 40 may be implemented not only as a cart-type device but also as a portable device. Examples of portable ultrasound imaging devices include a smartphone, a laptop computer, a Personal Digital Assistant (PDA), or a tablet PC including a probe and an application, but are not limited thereto. The ultrasound imaging device 40 may also be implemented integrally with a probe.

The probe 20 may include a wired probe that is connected to the ultrasound imaging device 40 by wire and communicates with the ultrasound imaging device 40 by wire, a wireless probe that is connected wirelessly to the ultrasound imaging device 40 and communicates wirelessly with the ultrasound imaging device 40, and/or a hybrid probe that is connected to the ultrasound imaging device 40 by wire or wirelessly and communicates with the ultrasound imaging device 40 by wire or wirelessly.

According to various embodiments of the present disclosure, as illustrated in FIG. 1A, the ultrasound imaging device 40 may include an ultrasound transceiver module 110, and as illustrated in FIG. 1B, the probe 20 may include an ultrasound transceiver module 110. According to various embodiments of the present disclosure, the ultrasound imaging device 40 and the probe 20 may include an ultrasound transceiver module 110, respectively.

According to various embodiments of the present disclosure, the probe 20 may further include at least one of an image processor 130, a display 140, or an input interface 170 or a combination thereof. In the present disclosure, a description of the ultrasound transceiver module 110, the image processor 130, the display 140, or the input interface 170 included in the ultrasound imaging device 40 may also be applied to the ultrasound transceiver module 110, the image processor 130, the display 140, or the input interface 170 included in the probe 20.

FIG. 1A is a block diagram illustrating a configuration of the ultrasound imaging system 100 when the probe 20 is a wired probe or a hybrid probe.

The probe 20 may include a plurality of transducers. The plurality of transducers may be arranged in a predetermined arrangement to be implemented as a transducer array. The transducer array may correspond to a one-dimensional (1D) array or a two-dimensional (2D) array. The plurality of transducers may transmit an ultrasonic signal to a target object (also referred to as object) 10 according to a transmission signal applied from a transmission module 113. The plurality of transducers may receive the ultrasonic signal (echo signal) reflected from the target object 10 and form a reception signal. In addition, the probe 20 may be implemented to be integrated with the ultrasound imaging device 40 or may be implemented as a separate type connected to the ultrasound imaging device 40 by a wire. In addition, the ultrasound imaging device 40 may be connected to one or a plurality of probes 20 depending on the implementation form.

When the probe 20 is a wired probe or a hybrid probe, the probe 20 may include a cable and a connector connectable to a connector of the ultrasound imaging device 40.

The probe 20 according to the present disclosure may be implemented as a two-dimensional probe. When the probe 20 is implemented as a two-dimensional probe, the plurality of transducers included in the probe 20 may be arranged two-dimensionally to form a two-dimensional transducer array.

For example, the two-dimensional transducer array may be a form in which a plurality of sub-arrays, which includes a plurality of transducers arranged in a first direction, is arranged in a second direction different from the first direction.

In addition, when the probe 20 according to one embodiment of the present disclosure is implemented as a two-dimensional probe, the ultrasound transceiver module 110 may include at least one of an analog beamformer or a digital beamformer. In addition, according to one embodiment of the present disclosure, the two-dimensional probe may include at least one of an analog beamformer or a digital beamformer or a combination thereof depending on the implementation form.

A processor 120 controls the transmission module 113 to form a transmission signal to be applied to each transducer 117 by considering positions and focus points of the plurality of transducers included in the probe 20.

The processor 120 may control a receiving module 115 to generate ultrasound data by performing analog-to-digital conversion on a reception signal received from the probe 20, and adding up the digital reception signals by considering the positions and focus points of the plurality of transducers.

When the probe 20 is implemented as a two-dimensional probe, the processor 120 may calculate a time delay value for digital beamforming for each sub-array of a plurality of sub-arrays included in the two-dimensional transducer array. In addition, the processor 120 may calculate a time delay value for analog beamforming for each transducer included in any one of the plurality of sub-arrays. The processor 120 may control an analog beamformer and a digital beamformer to form a transmission signal to be applied to each of the plurality of transducers according to the time delay values for the analog beamforming and the time delay values for the digital beamforming. In addition, the processor 120 may control the analog beamformer to add up signals, which are received from the plurality of transducers, for each sub-array according to the time delay value for the analog beamforming. In addition, the processor 120 may control the ultrasound transceiver module 110 to perform analog-to-digital conversion on the signal that is added up for each sub-array. Additionally, the processor 120 may control the digital beamformer to generate ultrasound data by adding up digitally converted signals according to the time delay value for the digital beamforming.

The image processor 130 may generate or process an ultrasound image by using the generated ultrasound data.

The display 140 may display the generated ultrasound image and various information processed by the ultrasound imaging device 40 or the probe 20. The probe 20 or the ultrasound imaging device 40 may include one or more displays 140 depending on the implementation form. In addition, the display 140 may include a touch panel or a touch screen. In addition, the display 140 may include a flexible display.

The processor 120 may control an overall operation of the ultrasound imaging device 40 and may control an operation of components of the ultrasound imaging device 40. The processor 120 may perform or control various operations or functions of the ultrasound imaging device 40 by executing programs or instructions stored in a memory 150. In addition, the processor 120 may receive a control signal from the input interface 170 or an external device, and control the operation of the ultrasound imaging device 40.

The ultrasound imaging device 40 may include a communication module 160 and may be connected to and communicate with an external device (for example, the probe 20, a server, a medical device, a portable device (smartphone, tablet PC, wearable device, and the like)) through the communication module 160.

The communication module 160 may include one or more components configured to allow communication with an external device. The communication module 160 may include at least one of a short-range communication module, a wired communication module, or a wireless communication module.

The communication module 160 may receive a control signal or data from an external device. The processor 120 may control an operation of the ultrasound imaging device 40 according to a control signal received through the communication module 160. In addition, the processor 120 may transmit a control signal to the external device through the communication module 160 and control the external device according to the transmitted control signal. The external device may operate according to the control signal received from the ultrasound imaging device 40 or process data received from the ultrasound imaging device 40.

A program or an application related to the ultrasound imaging device 40 may be installed on the external device. The program or application installed on the external device may control the ultrasound imaging device 40 or operate according to a control signal or data received from the ultrasound imaging device 40.

The external device may receive or download a program or application related to the ultrasound imaging device 40 from the ultrasound imaging device 40, the probe 20, or the server, and install and execute the program or application on the external device. The ultrasound imaging device 40, the probe 20, or the server that provides the program or application may include a recording medium that stores instructions, commands, installation files, executable files, or related data of the program or application. The external device may also be sold with the program or application installed thereon.

The memory 150 may store various data or programs for driving and controlling the ultrasound imaging device 40, input/output ultrasound data, ultrasound images, and the like.

The input interface 170 may receive user input for controlling the ultrasound imaging device 40. For example, the user input may include input for operating a button, a keypad, a mouse, a trackball, a jog switch, a knob, and the like, input for touching a touchpad or a touch screen, voice input, motion input, biometric information input (for example, iris recognition, fingerprint recognition, and the like), and the like, but is not limited thereto.

FIG. 1B illustrates a control block diagram of the ultrasound imaging system 100 when the probe 20 is a wireless probe or a hybrid probe.

According to various embodiments of the present disclosure, an ultrasound imaging device 40 illustrated in FIG. 1B may be replaced with the ultrasound imaging device 40 described with reference to FIG. 1A.

According to various embodiments of the present disclosure, the probe 20 illustrated in FIG. 1A may be replaced with a probe 20 to be described with reference to FIG. 1B.

The probe 20 may include a display 112, a transmission module 113, a battery 114, a receiving module 115, a charging module 116, a transducer 117, an input interface 109, a processor 118, and a communication module 119. In FIG. 1B, it is illustrated that the probe 20 includes both the transmission module 113 and the receiving module 115, but depending on the implementation form, the probe 20 may include only a portion of the configuration of the transmission module 113 and the receiving module 115, and a portion of the configuration of the transmission module 113 and the receiving module 115 may be included in the ultrasound imaging device 40. In addition, according to one embodiment of the present disclosure, the probe 20 may further include an image processor 130.

The transducer 117 may include a plurality of transducers. The plurality of transducers may be arranged in a predetermined arrangement to be implemented as a transducer array. The transducer array may correspond to a one-dimensional (1D) array or a two-dimensional (2D) array. The plurality of transducers may transmit ultrasonic signals to a target object 10 according to a transmission signal applied from the transmission module 113. In addition, the plurality of transducers may receive ultrasonic signals reflected from the target object 10 and form or generate electrical reception signals.

The charging module 116 may charge the battery 114. The charging module 116 may receive power from an external source. According to one embodiment of the present disclosure, the charging module 116 may receive power wirelessly. Further, according to one embodiment of the present disclosure, the charging module 116 may also receive power wired. The charging module 116 may transmit the received power to the battery 114.

The processor 118 controls the transmission module 113 to generate or form a transmission signal to be applied to each of the plurality of transducers by considering the positions and focus points of the plurality of transducers.

The processor 118 may control the receiving module 115 to generate ultrasound data by performing analog-to-digital conversion on a reception signal received from the transducer 117, and adding up the digital reception signals by considering the positions and focus points of

the plurality of transducers. According to one embodiment of the present disclosure, when the probe 120 includes the image processor 130, it is possible to generate an ultrasound image by using the generated ultrasound data.

When the probe 20 is implemented as a two-dimensional probe, the processor 118 may calculate a time delay value for digital beamforming for each sub-array of a plurality of sub-arrays included in the two-dimensional transducer array. In addition, the processor 118 may calculate a time delay value for analog beamforming for each transducer included in any one of the plurality of sub-arrays. The processor 118 may control an analog beamformer and a digital beamformer to form a transmission signal to be applied to each of the plurality of transducers according to the time delay value for the analog beamforming and the time delay value for the digital beamforming. In addition, the processor 118 may control the analog beamformer to add up signals, which are received from the plurality of transducers, for each sub-array according to the time delay value for analog beamforming. In addition, the processor 118 may control the ultrasound transceiver module 110 to perform analog-to-digital conversion on the signal that is added up for each sub-array. Additionally, the processor 118 may control the digital beamformer to generate ultrasound data by adding up digitally converted signals according to the time delay value for the digital beamforming.

The processor 118 may control an overall operation of the probe 20 and may control operation of components of the probe 20. The processor 118 may perform or control various operations or functions of the probe 20 by executing programs or instructions stored in a memory 111. In addition, the processor 118 may receive a control signal from an input interface 109 or an external device (for example, the ultrasound imaging device 40) and control the

operation of the probe 20. Further, the processor 118 may receive a control signal from the input interface 109 or an external device and control the operation of the probe 20. The input interface 109 may receive user input for controlling the probe 20. For example, the user input may include input for operating a button, a keypad, a mouse, a trackball, a jog switch, a knob, and the like, input for touching a touchpad or touch screen, voice input, motion input, biometric information input (for example, iris recognition, fingerprint recognition, and the like), and the like, but is not limited thereto.

The display 112 may display an ultrasound image generated by the probe 20, an ultrasound image generated by processing ultrasound data generated by the probe 20, an ultrasound image received from the ultrasound imaging device 40, or various information processed by the ultrasonic imaging system 100. In addition, the display 112 may further display status information of the probe 20. The status information of the probe 20 may include at least one of device information of the probe 20, battery status information of the probe 20, frequency band information of the probe 20, output information of the probe 20, information on the presence or absence of abnormality of the probe 20, setting information of the probe 20, or temperature information of the probe 20.

The probe 20 may include one or more displays 112 depending on the implementation form. In addition, the display 112 may include a touch panel or a touch screen. In addition, the display 112 may include a flexible display.

The communication module 119 may wirelessly transmit the generated ultrasound data or ultrasound image to the ultrasound imaging device 40 via a wireless network. In addition, the communication module 119 may receive control signals and data from the ultrasound imaging device 40.

The ultrasound imaging device 40 may receive ultrasound data or ultrasound image from the probe 20.

According to one embodiment of the present disclosure, when the probe 20 includes the image processor 130 configured to generate an ultrasound image using ultrasound data, the probe 20 may transmit the ultrasound data or the ultrasound image generated by the image processor 130 to the ultrasound imaging device 40.

According to one embodiment of the present disclosure, when the probe 20 does not include the image processor 130 configured to generate an ultrasound image using ultrasound data, the probe 20 may transmit the ultrasound data to the ultrasound imaging device 40. The ultrasound data may include ultrasound raw data, and the ultrasound image may represent ultrasound image data.

The ultrasound imaging device 40 may include the processor 120, the image processor 130, the display 140, the memory 150, the communication module 160, and the input interface 170.

The image processor 130 generates or processes an ultrasound image using ultrasound data received from the probe 20.

The display 140 may display an ultrasound image received from the probe 20, an ultrasound image generated by processing ultrasound data received from the probe 20, or various information processed in the ultrasound imaging system 100. The ultrasound imaging device 40 may include one or more displays 140 depending on the implementation type. In addition, the display 140 may include a touch panel or a touch screen. In addition, the display 140 may include a flexible display.

The processor 120 may control the overall operation of the ultrasound imaging device 40 and may control the operation of components of the ultrasound imaging device 40. The processor 120 may execute a program or application stored in the memory 150 to perform or control various operations or functions of the ultrasound imaging device 40. In addition, the processor 120 may receive a control signal from the input interface 170 or an external device and control the operation of the ultrasound imaging device 40.

The ultrasound imaging device 40 may include the communication module 160 and may be connected to and communicate with an external device (for example, the probe 20, a server, a medical device, a portable device (smartphone, tablet PC, wearable device, and the like)) through the communication module 160.

The communication module 160 may include one or more components configured to allow communication with an external device. The communication module 160 may include at least one of a short-range communication module, a wired communication module, or a wireless communication module.

The communication module 160 of the ultrasound imaging device 40 and the communication module 119 of the probe 20 may communicate using a network or a short-range wireless communication method. For example, the communication module 160 of the ultrasound imaging device 40 and the communication module 119 of the probe 20 may communicate using any one of wireless data communication methods including Wireless LAN, Wi-Fi, Bluetooth, Zigbee, Wi-Fi Direct (WFD), Infrared Data Association (IrDA), Bluetooth LowEnergy (BLE), Near Field Communication (NFC), Wireless Broadband Internet (Wibro), World Interoperability for Microwave Access (WiMAX), Shared Wireless Access Protocol (SWAP), Wireless Gigabit Alliance (WiGig), RF communication, or 60 GHz millimeter wave (mm Wave) short-range communication.

For the communication, the communication module 160 of the ultrasound imaging device 40 and the communication module 119 of the probe 20 may include at least one of a wireless LAN communication module, a Wi-Fi communication module, a Bluetooth communication module, a Zigbee communication module, a Wi-Fi Direct (WFD) communication module, an infrared Data Association (IrDA) communication module, a Bluetooth LowEnergy (BLE) communication module, a Near Field Communication (NFC) communication module, a Wireless Broadband Internet (Wibro) communication module, a World Interoperability for Microwave Access (WiMAX) communication module, a Shared Wireless Access Protocol (SWAP) communication module, a Wireless Gigabit Alliance (WiGig) communication module, an RF communication module, or a 60 GHz millimeter wave (mm Wave) short-range communication module.

According to one embodiment of the present disclosure, the probe 20 may transmit device information (for example, identification (ID) information) of the probe 20 to the ultrasound imaging device 40 using a first communication method (for example, BLE) and may be wirelessly paired with the ultrasound imaging device 40. In addition, the probe 20 may transmit ultrasound data and/or ultrasound images to the paired ultrasound imaging device 40.

The device information of the probe 20 may include various information related to a serial number, model name, or battery status of the probe 20.

The ultrasound imaging device 40 may receive device information (for example, ID information) of the probe 20 from the probe 20 using the first communication method (for example, BLE) and may be wirelessly paired with the probe 20. In addition, the ultrasound imaging device 40 may transmit an activation signal to the paired probe 20 and receive ultrasound data and/or ultrasound images from the probe 20. At this time, the activation signal may include a signal for controlling the operation of the probe 20.

According to one embodiment of the present disclosure, the probe 20 may transmit device information (for example, ID information) of the probe 20 to the ultrasound imaging device 40 using the first communication method (for example, BLE) and may be wirelessly paired with the ultrasound imaging device 40. In addition, the probe 20 may transmit ultrasound data and/or ultrasound images to the ultrasound imaging device 40 paired by the first communication method using a second communication method (for example, 60 GHz millimeter wave, and Wi-Fi).

The ultrasound imaging device 40 may receive device information (for example, ID information) of the probe 20 from the probe 20 using the first communication method (for example, BLE) and may be wirelessly paired with the probe 20. In addition, the ultrasound imaging device 40 may transmit an activation signal to the paired probe 20 and receive ultrasound data and/or ultrasound images from the probe 20 using the second communication method (for example, 60 GHz millimeter wave, and Wi-Fi).

According to one embodiment of the present disclosure, the first communication method used to pair the probe 20 and the ultrasound imaging device 40 with each other may have a lower frequency band than a frequency band of the second communication method used to transmit ultrasound data and/or ultrasound images from the probe 20 to the ultrasound imaging device 40.

The ultrasound imaging device 40 may display User Interfaces (UIs) indicating the device information of the probe 20. For example, the display 140 may display a UI indicating ID information of the wireless ultrasonic probe 20, a pairing method indicating a pairing method with the probe 20, a data communication status between the probe 20 and the ultrasound imaging device 40, a method of performing data communication with the ultrasound imaging device 40, or a battery status of the probe 20.

When the probe 20 includes the display 112, the display 112 of the probe 20 may display a UI indicating device information of the probe 20. For example, the display 112 may display a UI indicating ID information of the wireless ultrasound probe 20, a pairing method indicating a pairing method with the probe 20, a data communication status between the probe 20 and the ultrasound imaging device 40, a method of performing data communication with the ultrasound imaging device 40, or a battery status of the probe 20.

The communication module 160 may receive control signals or data from an external device. The processor 120 may control the operation of the ultrasound imaging device 40 according to the control signal received through the communication module 160.

In addition, the processor 120 may transmit a control signal to an external device through the communication module 160 and control the external device according to the transmitted control signal. The external device may operate according to the control signal received from the ultrasound imaging device 40 or process data received from the ultrasound imaging device 40.

The external device may receive or download a program or application related to the ultrasound imaging device 40 from the ultrasound imaging device 40, the probe 20, or a server, and install and execute the program or application on the external device. The ultrasound imaging device 40, the probe 20, or the server that provides the program or application may include a recording medium that stores instructions, commands, installation files, executable files, or related data of the program or application. The external device may also be sold with the program or application installed thereon.

The memory 150 may store various data or programs for driving and controlling the ultrasound imaging device 40, input/output ultrasound data, ultrasound images, and the like.

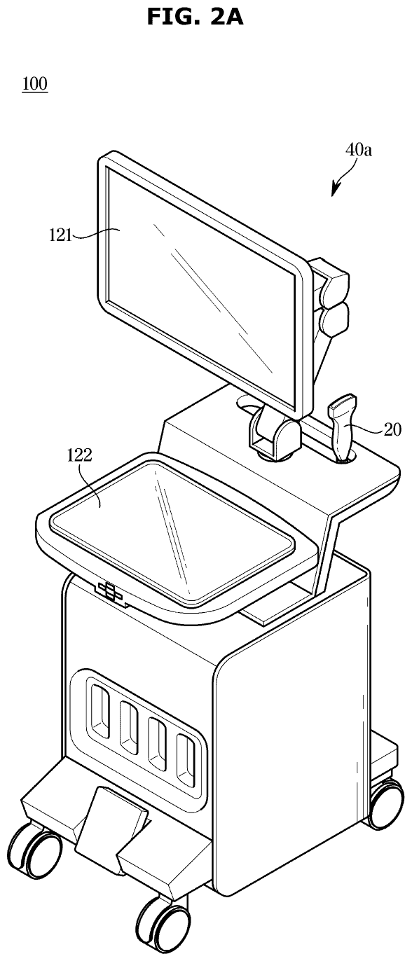

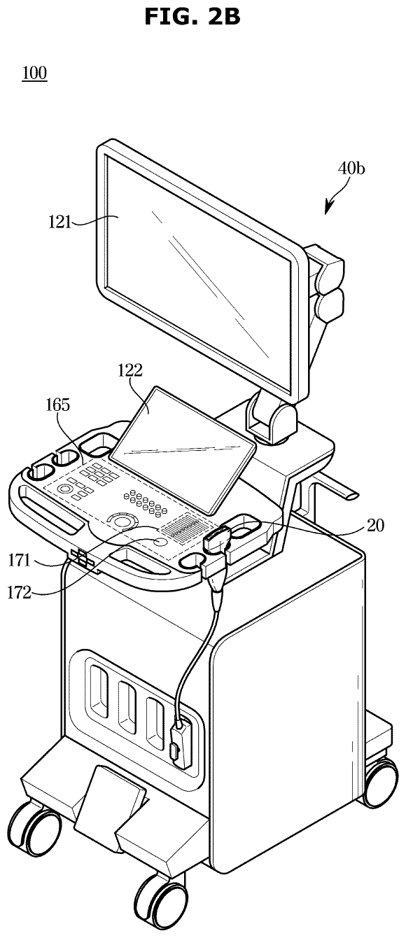

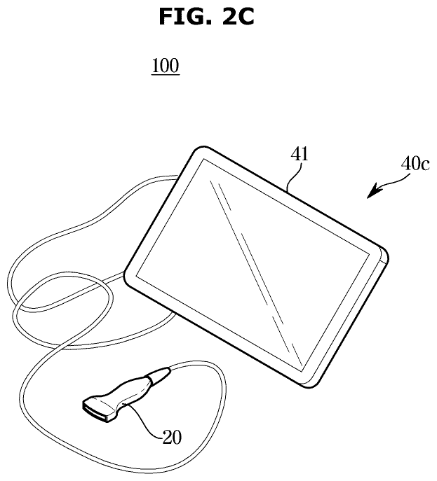

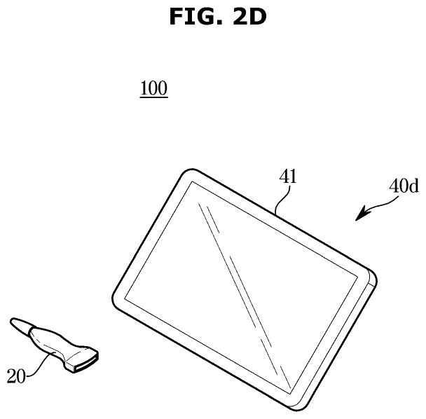

Examples of the ultrasound imaging system 100 according to one embodiment of the present disclosure will be described below with reference to FIGS. 2A, 2B, 2C, and 2D.

FIGS. 2A, 2B, 2C, and 2D are views illustrating an ultrasound imaging device according to one embodiment of the present disclosure.

Referring to FIGS. 2A and 2B, ultrasound imaging devices 40a and 40b may include a main display 121 and a sub-display 122. The main display 121 and the sub-display 122 may correspond to the display 140 of FIGS. 1A and 1B. At least one of the main display 121 or the sub-display 122 may be implemented as a touch screen. At least one of the main display 121 or the sub-display 122 may display an ultrasound image or various information processed in the ultrasound imaging devices 40a and 40b. In addition, at least one of the main display 121 or the sub-display 122 may be implemented as a touch screen and may provide a Graphical User Interface (GUI) to receive data for controlling the ultrasound imaging devices 40a and 40b from a user. For example, the main display 121 may display an ultrasound image, and the sub-display 122 may display a control panel in the form of a GUI for controlling the display of the ultrasound image. The sub-display 122 may receive data for controlling the display of the image through the control panel displayed in the form of a GUI. For example, a Time Gain Compensation (TGC) button, a Lateral Gain Compensation (LGC) button, a Freeze button, a trackball, a jog switch, or a knob may be provided as a GUI on the sub-display 122.

The ultrasound imaging devices 40a and 40b may control the display of the ultrasound image displayed on the main display 121 using the input control data. In addition, the ultrasound imaging devices 40a and 40b may be connected to the probe 20 by wire or wirelessly to transmit and receive ultrasonic signals to and from the target object.

Referring to FIG. 2B, the ultrasound imaging device 40b may further include a control panel 165 in addition to the main display 121 and the sub-display 122. The control panel 165 may include a button, a trackball, a jog switch, a knob, and the like, and may receive data for controlling the ultrasound imaging device 40b from a user. For example, the control panel 165 may include a TGC button 171, a Freeze button 172, and the like. The TGC button 171 is a button for setting a TGC value according to a depth of an ultrasound image. In addition, when the ultrasound imaging device 40b detects the input of the Freeze button 172 while scanning an ultrasound image, the ultrasound imaging device 40b may maintain a state in which a frame image at the corresponding time is displayed, capture the frame image at the corresponding time, or store the frame image at the corresponding time.

Meanwhile, the button, the trackball, the jog switch, the knob, and the like included in the control panel 165 may be provided as a GUI on the main display 121 or the sub-display 122. In addition, the ultrasound imaging devices 40a and 40b may be connected to the probe 20 to transmit and receive ultrasonic signals to a target object.

In addition, the ultrasound imaging devices 40a and 40b may include various types of input/output interfaces such as speakers, light emitting diodes (LEDs), and vibration devices. For example, the ultrasound imaging devices 40a and 40b may output various information in the form of graphics, sounds, or vibrations through the input/output interface. In addition, the ultrasound imaging devices 40a and 40b may output various notifications or data through the input/output interface.

Referring to FIGS. 2C and 2D, ultrasound imaging devices 40c and 40d may also be implemented in a portable form. Examples of the portable ultrasound imaging devices 40c and 40d include a smart phone, a laptop computer, a PDA, or a tablet PC which includes a probe and an application, but is not limited to.

The ultrasound imaging device 40c may include a main body 41. Referring to FIG. 2C, a probe 20 may be connected to one side of the main body 41 by a wire. For this, the main body 41 may include a detachable connection terminal for a cable connected to the probe 20. The probe 20 may include a cable including a connection terminal connectable to the main body 41.

Referring to FIG. 2D, a probe 20 may be wirelessly connected to an ultrasound imaging device 40d. The main body 41 may include an input/output interface (for example, a touch screen). The input/output interface may display ultrasound images, various information processed by the ultrasound imaging device, or a GUI.

The ultrasound imaging device 40d and the probe 20 may establish communication or be paired using short-range wireless communication. For example, the ultrasound imaging device 40d and the probe 20 may perform communication using Bluetooth, BLE, Wi-Fi, or Wi-Fi Direct.

The ultrasound imaging devices 40c and 40d may execute a program or application related to the probe 20, control the probe 20, and output information related to the probe 20. The ultrasound imaging devices 40c and 40d may communicate with a predetermined server and perform operations related to the probe 20. The probe 20 may be registered on the ultrasound imaging devices 40c and 40d or registered on a predetermined server. The ultrasound imaging devices 40c and 40d may communicate with the registered probe 20 and perform operations related to the probe 20.

In addition, the ultrasound imaging devices 40c and 40d may include various types of input/output interfaces such as speakers, LEDs, and vibration devices. For example, the ultrasound imaging devices 40c and 40d may output various information in the form of graphics, sounds, or vibrations through the input/output interface. In addition, the ultrasound imaging devices 40c and 40d may output various notifications or data through the input/output interface.

According to one embodiment the present disclosure, the ultrasound imaging device 40a, 40b, 40c, or 40d may process an ultrasound image or obtain additional information from an ultrasound image using an artificial intelligence (AI) model. According to one embodiment of the present disclosure, the ultrasound imaging device 40a, 40b, 40c, or 40d may generate an ultrasound image or perform processing such as correction, image quality improvement, encoding, or decoding on an ultrasound image using the AI model. In addition, according to one embodiment of the present disclosure, the ultrasound imaging device 40a, 40b, 40c, or 40d may perform processing, such as baseline definition, anatomical information acquisition, lesion information acquisition, surface extraction, boundary definition, length measurement, area measurement, volume measurement, or annotation generation, from an ultrasound image using the AI model.

The AI model may be installed on the ultrasound imaging device 40a, 40b, 40c, or 40d or on a server.

The AI models may be implemented using various artificial neural network or deep neural network. Further, the AI model may be trained and generated using various machine learning or deep learning algorithms. For example, the AI model may be implemented using models such as convolutional neural network (CNN), recurrent neural network (RNN), generative adversarial network (GAN), and long short-term memory (LSTM).

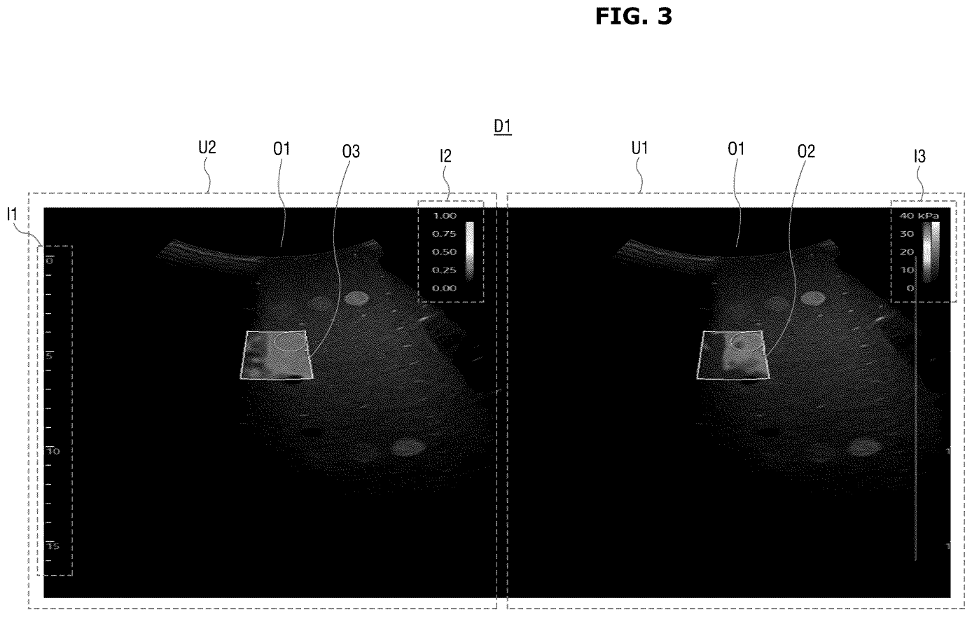

FIG. 3 is a view illustrating a configuration of an image frame displayed on a display device of the ultrasound imaging system according to one embodiment.

In one embodiment, the display (for example, the display 140 of FIG. 1A, the display 140 or the display 112 of FIG. 1B, or the display 121 of FIGS. 2A and 2B, and the display 140 will be described as an example hereinafter) may simultaneously display various images, which are generated based on echo signals and shear wave tracking signals received from the probe 20, in a single image frame. An image frame corresponds to one complete ultrasound image obtained at a specific point in time. Each image frame is a snapshot representing a tissue state at that point in time, and a B-mode image, an elasticity image, and an elasticity reliability image may be temporally synchronized to constitute one frame. For example, referring to FIG. 3, an image frame D1 displayed on the display 140 may simultaneously display an ultrasound image O1, an elasticity image O2, and an elasticity reliability image O3.

The ultrasound image O1 may correspond to an ultrasound image generated based on an echo signal received from the probe 20. For example, the ultrasound image O1 may correspond to the B-mode image. The B-mode image may express an anatomical structure of a target object 10 as black and white brightness information. The ultrasound image O1 may provide basic image information used in general ultrasound diagnosis, and may be generated based on an echo signal resulting from a difference in acoustic impedance at a tissue boundary.

The elasticity image O2 may be generated based on a shear wave tracking signal received on the same time stamp as the echo signal from the probe 20.

Particularly, Acoustic Radiation Force Impulse (ARFI), corresponding to a high-intensity focused pulse, transmitted from the probe 20 may induce a minute mechanical displacement (approximately 10-40 micrometers) at a specific depth within the target object 10. In a process of restoring this displacement, a shear wave may be generated within the tissue, and the shear wave may propagate laterally perpendicular to a direction of compression.

The generated shear waves may propagate at different speeds depending on elastic properties of the tissue. The ultrasound imaging system 100 may track a propagation process of the shear waves in real time using an ultra-high-speed ultrasound imaging technique (up to 10,000 fps).

The ultrasound imaging system 100 may calculate an elastic modulus using the measured shear wave velocity (Vs). The ultrasound imaging system 100 may convert the calculated elastic modulus value into colors according to a predefined color map, thereby generating the elasticity image O2. Typically, in the elasticity image O2, hard tissue (high elastic modulus) may be displayed in shades of red, soft tissue (low elastic modulus) may be displayed in shades of blue, and intermediate hardness may be displayed in shades of green or yellow. However, the present disclosure is an example, and other colors may be used to distinguish and display the tissue in addition to the mentioned colors.

The elasticity reliability image O3 may be generated based on various signal quality parameters obtained from the shear wave tracking signals.

The ultrasound imaging system 100 may obtain the elasticity reliability image O3 indicating a reliability of an elastic value (that is, an elastic modulus) based on the received ultrasound echo signal. For example, the ultrasound imaging system 100 may determine at least one of a size of a transverse wave, a signal quality of a transverse wave, or a noise level of a transverse wave based on the received ultrasound echo signal. The ultrasound imaging system 100 may generate the elasticity reliability image O3 indicating the reliability of elastic modulus indicated by the reference elasticity image O2 based on the size of the transverse wave, the signal quality of the transverse wave, or the noise level of the transverse wave. For example, the ultrasound imaging system 100 may determine the reliability of the elastic value to be higher as the size of the induced transverse wave is larger, the signal quality of the transverse wave is higher, or the noise level of the transverse wave is smaller.

The ultrasound imaging system 100 may generate the elasticity reliability image O3 by converting an elasticity reliability value into colors according to the reliability level. Typically, a high reliability (for example, elasticity reliability>0.7) region may be displayed in dark green, an intermediate reliability (for example, 0.4<elasticity reliability<0.7) region may be displayed in light green or yellow, and a low reliability (for example, elasticity reliability<0.4) region may be displayed in orange or red. However, the present disclosure is an example, and other colors may be used to distinguish and display the regions in addition to the above-described colors.

According to one embodiment, registration of ultrasound image, elasticity image, and elasticity reliability image may be performed in a variety of methods.

For example, image registration may be performed using an overlay method. The most common of the overlay method is that superimposes another image (elasticity image or elasticity reliability image) on top of a reference image (ultrasound image) in a semi-transparent manner. Alpha blending may be used to control transparency. For example, transparency may typically be set to from 50 to 70%.

The display 140 may simultaneously display a first ultrasound image U1 and a second ultrasound image U2. That is, the image frame D1 displayed on the display 140 may include the first ultrasound image U1 and the second ultrasound image U2.

The first ultrasound image U1 may be generated by aligning the elasticity image O2 with the ultrasound image O1. Anatomical structural information of the ultrasound image may be maintained in grayscale, and the color information of the elasticity image may be overlaid with a specific transparency. Accordingly, it is possible to simultaneously identify a precise anatomical location and elasticity characteristics of a lesion, thereby providing useful information for the diagnosis of liver fibrosis, thyroid nodules, breast tumors, and other conditions.

The second ultrasound image U2 may be generated by aligning the elasticity reliability image O3 with the ultrasound image O1. The second ultrasound image U2 may display the elasticity measurement reliability in each unit region in real time, along with anatomical structural information, and thus it is possible to immediately assess the quality of the measurement results. Particularly, a region with low measurement reliability due to movement caused by breathing or heartbeat, or tissue inhomogeneity, may be easily identified.

Simultaneously displaying the first ultrasound image U1 and the second ultrasound image U2 may significantly enhance the clinical utility of elastography. A user can simultaneously check a lesion hardness distribution in the first ultrasound image on the right and assess the reliability of the corresponding measurement in the second ultrasound image on the left. The dual validation system may significantly reduce false positives and false negatives, and thus more accurate and reliable diagnoses may be possible.

In addition, the image frame may further include a plurality of indicators I1, I2, and I3.

For example, the image frame may further include a depth scale I1 indicating depth information of the image. The depth scale I1 may be typically located on the left or right side of the ultrasound image and may indicate a distance from a probe surface in centimeters (cm). Accordingly, it is possible to identify a precise depth of the lesion and thus it is possible to consider measurement conditions according to the depth when measuring elasticity.

As another example, the ultrasound image may include an elastic modulus color index I3 corresponding to the elasticity image O2. The elastic modulus color index I3 may indicate correlation between the elastic modulus value (kilopascals (kPa)) and the corresponding color, and may typically be located at an upper end or a lateral side of the image. This color index allows a user to immediately identify the exact elastic modulus value represented by the color displayed in the image. The color index may be scaled according to the clinical application. For example, it may be set to a range from 0 to 40 kPa for liver diagnosis, and a range from 0 to 200 kPa for breast diagnosis.

As another example, the ultrasound image may include a reliability color index I2 corresponding to the elasticity reliability image O3. The reliability color index I2 may indicate a correlation between a reliability index (range from 0 to 1) and the corresponding color. For example, from 0 to 0.4 may be displayed in shades of red (low reliability), from 0.4 to 0.7 may be displayed in shades of yellow (intermediate reliability), and from 0.7 to 1.0 may be displayed in shades of green (high reliability). This allows a user to quantitatively assess the measurement reliability in each unit region and consider additional measurements or other diagnostic methods in regions with the low reliability.

Within each image frame, the ultrasound image O1, the elasticity image O2, and the elasticity reliability image O3 may be obtained at the same time stamp. This is because the echo signal and shear wave tracking signal are simultaneously received and processed by the probe 20. Due to the temporal synchronization, an accurate correspondence between the images may be secured, and momentary changes in the tissue's state may be precisely captured.

A cine image may correspond to an image that is sequentially obtained to capture dynamic changes in the tissue, such as changes in a patient's breathing cycle, heart rate, or probe pressure. The cine image may include a plurality of image frames. Individual image frames obtained at each point in time may be stored in chronological order, along with metadata (time information, acquisition conditions, and the like), to form the cine image.

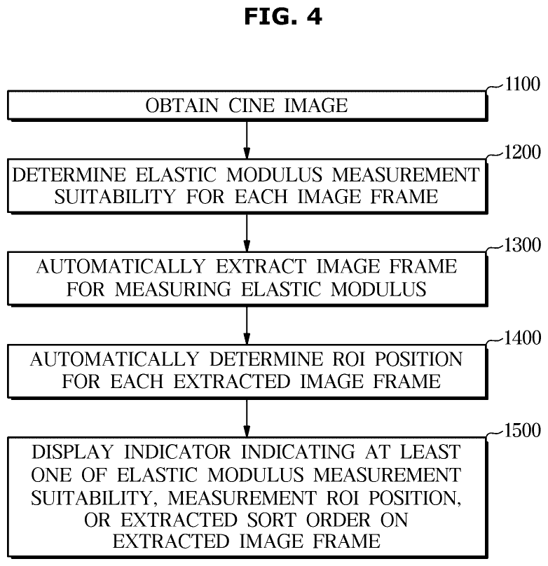

FIG. 4 is a flowchart illustrating a control method of the ultrasound imaging system according to one embodiment.

A control method of the ultrasound imaging system 100 according to one embodiment may include obtaining a cine image (1100).

The ultrasound imaging system 100 may obtain the cine image including a plurality of ultrasound image frames based on the echo signal and shear wave tracking signal received from the probe 20. Each ultrasound image frame may be configured in a form in which a B-mode image, an elasticity image, and a reliability measurement index (RMI) image are aligned at the same time stamp.

The control method of the ultrasound imaging system 100 according to one embodiment may include calculating an elastic modulus measurement suitability for each image frame (1200).

The ultrasound imaging system 100 may determine the elastic modulus measurement suitability by analyzing elasticity image data and elasticity reliability image data for each of the plurality of ultrasound image frames constituting the cine image.

The elastic modulus measurement suitability may be determined and displayed in real time simultaneously with image acquisition.

The elastic modulus measurement suitability may refer to a quantitative index indicating how suitable it is for measuring the elastic modulus in a specific ultrasound image frame. The elastic modulus measurement suitability may be expressed as a score value within a certain range by comprehensively analyzing the uniformity of the elasticity image and the reliability of the elasticity reliability image. For example, the elastic modulus measurement suitability may be expressed as an index of a specific shape that is greater than or equal to 1 and less than or equal to 5. By using indexed values, the measurement quality of each image frame may be objectively and consistently evaluated, and optimal measurement conditions may be automatically identified without relying on the user subjective judgment.

In this process, the measurement quality of each frame may be quantitatively evaluated by comprehensively considering the elasticity uniformity and the reliability index.

The control method of the ultrasound imaging system 100 according to one embodiment may include automatically extracting an image frame for measuring an elastic modulus (1300).

The ultrasound imaging system 100 may automatically select image frames having a suitability greater than or equal to a reference value based on the calculated elastic modulus measurement suitability. At this time, a threshold value for automatically extracting image frames may be preset and stored in the memory 150. In addition, according to various embodiments, the threshold value may be changed based on user input received through the input interface 170, and the changed threshold value may be stored in the memory 150 and immediately applied to extract new suitable frames. Accordingly, it is possible to exclude frames that are unsuitable for measurement and it is possible to extract only frames that are suitable for measurement with the high reliability. Accordingly, consistent examination quality may be guaranteed without relying on skill or experience of an examiner, thereby achieving standardization of diagnostic accuracy and improvement of reproducibility.

Particularly, automatically extracting an image frame for the elastic modulus measurement may include automatically extracting a plurality of image frames for the elastic modulus measurement based on whether the elastic modulus measurement suitability is greater than or equal to the reference value. For example, the ultrasound imaging system 100 may automatically extract an image frame for the elastic modulus measurement based on the elastic modulus measurement suitability of the image frame being greater than or equal to the reference value.

The ultrasound imaging system 100 may display that there is no a plurality of image frames extracted for the elastic modulus measurement, through the user interface 170 based on the elastic modulus measurement suitability of all of image frames being less than the reference value. The information that there is no the plurality of image frames extracted for the elastic modulus measurement may be displayed in various sensory information forms. In addition, the ultrasound imaging system 100 may provide a guide for inducing resetting of the elastic modulus measurement suitability threshold value through the user interface 170 based on the elastic modulus measurement suitability of all of image frames being less than the reference value.

The control method of the ultrasound imaging system 100 according to one embodiment may include automatically determining a measurement region of interest (ROI) position for each extracted image frame (1400).

The ultrasound imaging system 100 may automatically determine an optimal measurement ROI position based on elasticity image data and elasticity reliability image data for each extracted image frame. In this process, a position where the most accurate measurement is possible may be identified by comprehensively analyzing the elasticity uniformity and reliability for each unit region.

In the present disclosure, a ROI may refer to a general region of interest to a user or system in an ultrasound image. The ROI may be set in various sizes and shapes depending on the diagnostic purpose, and may represent a target region in which image analysis, measurement, or processing is performed.

On the other hand, a measurement ROI may be a region particularly set for measuring an elastic modulus, and may represent a specific measurement point for obtaining a quantitative elastic modulus value. The measurement ROI may be generally implemented in a circular or rectangular shape, and size and shape thereof may be automatically optimized in consideration of elasticity uniformity and reliability. In addition, according to various embodiments, the size and shape of the measurement ROI may be changed based on user input received through the input interface 170. The elastic modulus value calculated within the measurement ROI may be used as a quantitative index directly utilized in the clinical diagnosis.

The control method of the ultrasound imaging system 100 according to one embodiment may include displaying an indicator indicating at least one of elastic modulus measurement suitability, measurement ROI position, or extracted image frame sort order in an extracted image frame (1500).

The ultrasound imaging system 100 may provide various indicators indicating elastic modulus measurement suitability, measurement ROI position, or sort order while displaying one of the extracted image frames on the display 140. Accordingly, a user can check and evaluate the reliability and quality of the measurement result in real time. An indicator for indicating the elastic modulus measurement suitability may be referred to as a first indicator. An indicator for indicating the measurement ROI position may be referred to as a second indicator. An indicator for indicating the sort order may be referred to as a third indicator. Hereinafter the first indicator, the second indicator, and the third indicator will be described in detail.

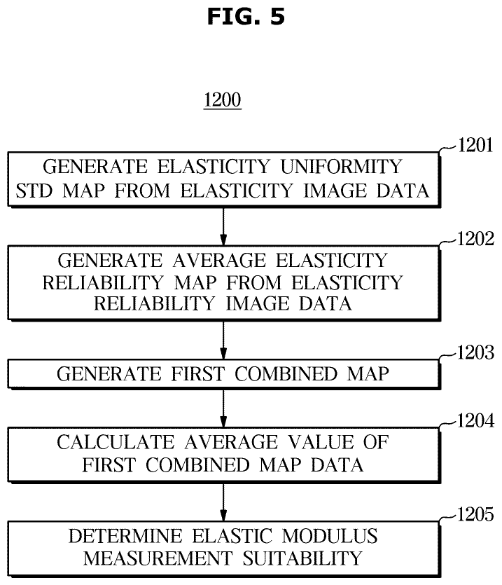

FIG. 5 is a flowchart illustrating a method for calculating an elastic modulus measurement suitability for each image frame according to one embodiment.

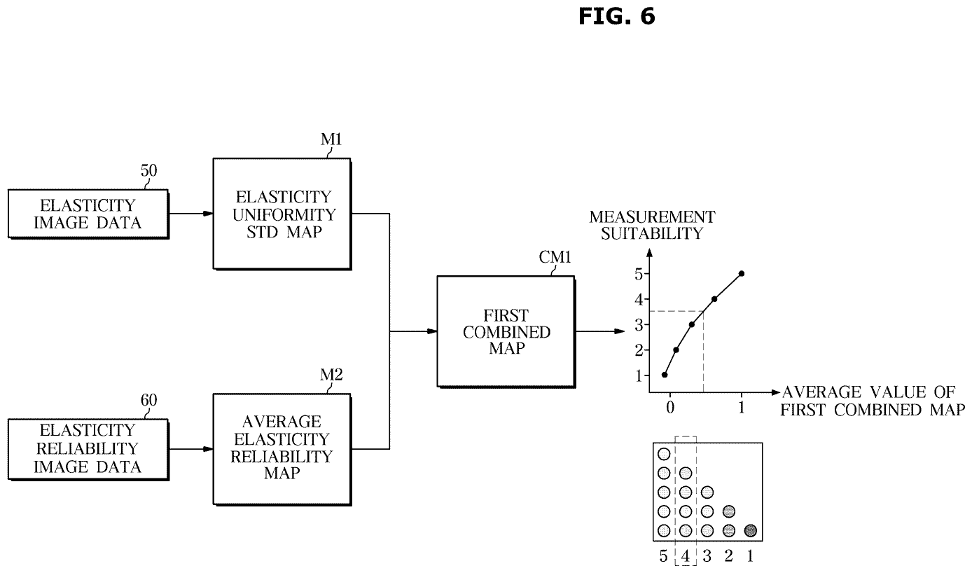

FIG. 6 is a block diagram briefly illustrating a configuration and processing of data input and output in a process of determining the elastic modulus measurement suitability.

A method for calculating an elastic modulus measurement suitability according to one embodiment (1200) may include generating an elasticity uniformity STD map (1201).

The ultrasound imaging system 100 may calculate an elasticity uniformity standard deviation for each unit region from elasticity image data 50 of each of the plurality of image frames. The elasticity uniformity standard deviation for each unit region may represent an index indicating how uniformly elasticity coefficient values are distributed within a kernel of a predetermined size (for example, 5×5 or 7×7 pixels) in each image frame.

The ultrasound imaging system 100 may also calculate an elastic modulus standard deviation of the surrounding region at each location while scanning the entire elasticity image in a sliding window manner.

The ultrasound imaging system 100 may generate an elasticity uniformity STD map M1 by normalizing and inverting the calculated standard deviation value. In this process, a region with a small standard deviation (a uniform region) may be converted to a high value, and a region with a large standard deviation (an uneven region) may be converted to a low value. Normalization may be performed using a formula: Uniformity=1−(standard deviation/maximum possible standard deviation), and a final value may be expressed in a range from 0 to 1. The ultrasound imaging system 100 may generate the STD map M1 by spatially mapping the final value.

The method for calculating the elastic modulus measurement suitability according to one embodiment (1200) may include generating an average elasticity reliability map for each unit region M2 from elasticity reliability image data 60 of each of the plurality of image frames (1202).

The elasticity reliability image data may be composed of values ranging from 0 to 1 representing the elastic measurement reliability at each pixel. The ultrasound imaging system 100 may calculate the elasticity reliability average value of each unit region by applying a kernel of the same size as the elasticity uniformity STD map generation.

The ultrasound imaging system 100 may apply a morphology operation (Opening) to the calculated average elasticity reliability map M2 to remove noise and mitigate the influence of low-reliability region. The morphology opening operation is a process that performs dilation after erosion, and may provide an effect of removing isolated noise pixels and smoothing boundaries. Accordingly, it is possible to minimize the effects of temporary signal quality degradation or motion artifacts.

The method for calculating the elastic modulus measurement suitability according to one embodiment (1200) may include generating a first combined map CM1 (1203).

The ultrasound imaging system 100 may generate the first combined map by being synthesized with the average elasticity reliability map M2.

The ultrasound imaging system 100 may synthesize the representative elasticity uniformity STD map and the average elasticity reliability map in various methods. For example, the first combined map may be generated by multiplying pixel values at the same location by element-wise multiplication. In another embodiment, a weighted sum method may be used, and in this case, a formula: Combined_Map=α×STD_map+β×elasticity reliability_map (α+β=1) may be applied. Combined_Map may represent the first combined map, α may represent a weighted value of the STD map, β may represent a weighted value of the elasticity reliability map, STD_map may represent the representative elasticity uniformity STD map, and elasticity reliability_map may represent the average map.

The method for calculating the elastic modulus measurement suitability according to one embodiment (1200) may include calculating an average value of first combined map CM1 data (1204).

The ultrasound imaging system 100 may calculate an average value for all pixel values of the generated first combined map CM1. This average value may be used as an index of the overall measurement quality of the corresponding image frame.

The ultrasound imaging system 100 may apply various methods when calculating the average value of the first combined map. When using the arithmetic mean, it may be calculated by dividing the sum of all pixel values with the total number of pixels. When using the weighted mean, a higher weighted value may be assigned to the center of the image, or a weighted value may be applied only to pixels within a ROI. Additionally, the average value may be calculated by applying a trimmed mean method to minimize the influence of extreme values. For example, the average may be calculated by excluding the top 5% and bottom 5% of values.

The method for calculating the elastic modulus measurement suitability according to one embodiment (1200) may include determining an elastic modulus measurement suitability for each image frame (1205).

The ultrasound imaging system 100 may determine the elastic modulus measurement suitability by scoring the average value of the first combined map into an integer value between 1 and 5. For example, a range from 0.0 to 0.2 may be classified as 1 point, a range from 0.2 to 0.4 may be classified as 2 points, a range from 0.4 to 0.6 may be classified as 3 points, a range from 0.6 to 0.8 may be classified as 4 points, and a range from 0.8 to 1.0 may be classified as 5 points. Through the scoring process, the measurement quality of each image frame may be expressed as an integer value that may be intuitively identified. Referring to FIG. 6, the process of converting the average value of the first combined map into an integer value between 1 and 5 may be visually represented, and may be provided as a visual indicator distinguished by color according to each suitability level.

The ultrasound imaging system 100 may store the calculated elastic modulus measurement suitability in the memory 150 and utilize the calculated elastic modulus measurement suitability for a subsequent frame selection process. Further, parameters of the suitability calculation algorithm may be adjusted based on user input received via the input interface 170. For example, a user can adjust a weight ratio of the STD map and the elasticity reliability map, and the settings values may be stored in the memory 150 and applied to subsequent examinations.

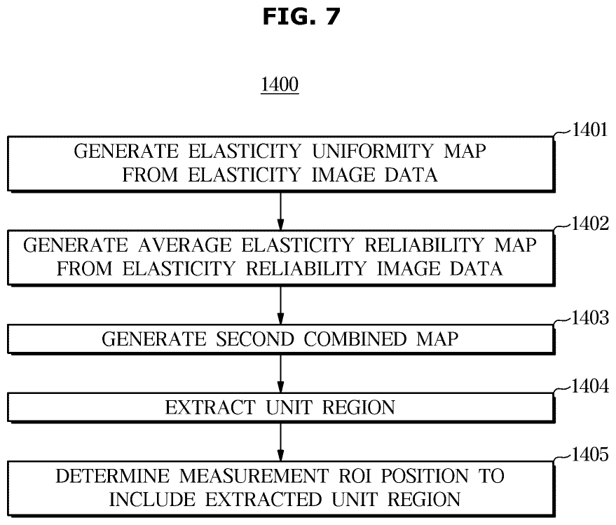

FIG. 7 is a flowchart illustrating a method for automatically determining a measurement region of interest (ROI) position for each extracted image frame according to one embodiment.

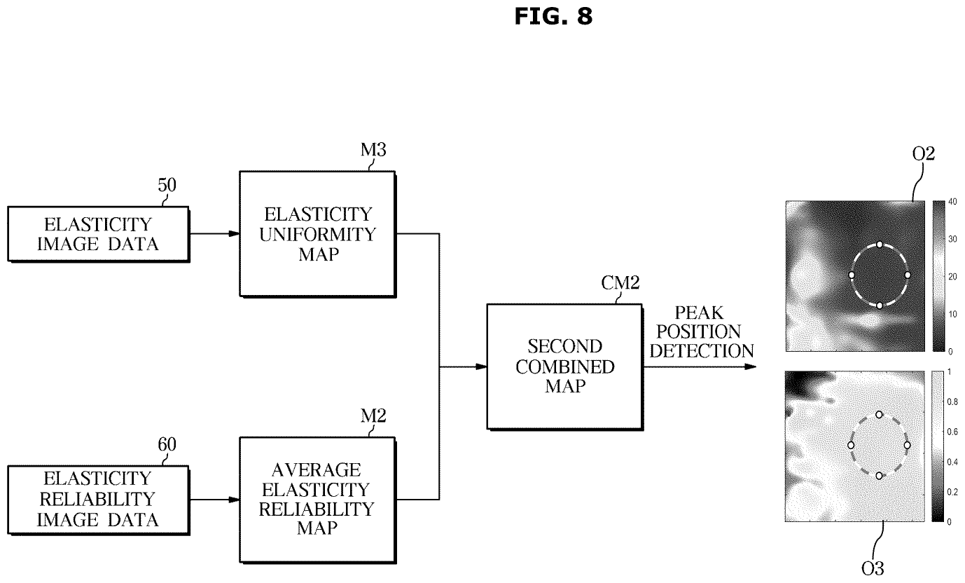

FIG. 8 is a block diagram briefly illustrating a configuration and processing of data that is input and output in the automatic determination of measurement ROI position.

A method for automatically determining a measurement region of interest (ROI) position according to one embodiment (1400) may include generating an elasticity uniformity map M3 by calculating an elasticity uniformity for each unit region from the elasticity image data 50 (1401).

The elasticity uniformity for each unit region may refer to an index of the consistency of elastic modulus values within a certain region (for example, 5×5, 7×7, or 9×9 pixels) surrounding each pixel. Unlike the suitability calculation for the entire frame described above, this may refer to a process of evaluating individual uniformity for each measurement ROI size.

For example, the ultrasound imaging system 100 may calculate an average of elastic modulus of a kernel region centered on each pixel in a sliding window manner.

The ultrasound imaging system 100 may generate the elasticity uniformity map M3 by spatially mapping the calculated elasticity uniformity values.

The method for automatically determining the measurement ROI position according to one embodiment (1400) may include generating the average elasticity reliability map M2 for each unit region from the elasticity reliability image data 60 (1402).

The average elasticity reliability for each unit region may refer to a result of calculating the average of elasticity reliability values in a kernel region of the same size around each pixel. The ultrasound imaging system 100 may ensure spatial consistency by using the same kernel size as the elasticity uniformity calculation. The elasticity reliability value may be a continuous value in a range from 0 to 1, and may represent the elasticity measurement reliability at each position.

The elasticity reliability image data may be composed of values in a range from 0 to 1 representing the elastic measurement reliability at each pixel. The ultrasound imaging system 100 may calculate an elasticity reliability average value of each unit region by applying a kernel of the same size as the generation of the elasticity uniformity STD map.

The ultrasound imaging system 100 may apply a morphology operation (Opening) to the calculated average elasticity reliability map M2 to remove noise and mitigate the influence of low-reliability region. The morphology opening operation is a process that performs dilation after erosion, and may provide an effect of removing isolated noise pixels and smoothing boundaries. Accordingly, it is possible to minimize the effects of temporary signal quality degradation or motion artifacts.

The method for automatically determining the measurement ROI position according to one embodiment (1400) may include generating a second combined map (1403). The ultrasound imaging system 100 may synthesize the elasticity uniformity STD map and the average elasticity reliability map in various methods. For example, the second combined map CM2 may be generated by multiplying pixel values at the same position by element-wise multiplication. In addition, the ultrasound imaging system 100 may apply a first weighted value to the elasticity uniformity map M3 and a second weighted value to the average elasticity reliability map M2 to calculate a composite score for each unit region, and generate the second combined map CM2 on which the composite score is reflected.

The ultrasound imaging system 100 may determine the first and second weighted values based on a user identification number obtained from the user interface 170 or the communication interface. The weighted values applied to the elasticity uniformity and elasticity reliability may vary depending on the user. For example, one user may apply a high weight (0.7) to the elasticity uniformity, while another user may apply a high weight (0.7) to the elasticity reliability.

The composite score calculation may be implemented in various methods. The most common method is the weighted sum method, which may calculate the composite score using a formula: Combined_Score=w1×Uniformity+w2×Elasticity Reliability (w1+w2=1). Combined_Score may represent a composite score of each pixel, w1 may represent a weighted value of elasticity uniformity, w2 may represent a weighted value of elasticity reliability, Uniformity may represent an elasticity uniformity value of the pixel, and elasticity reliability may represent an elasticity reliability value of the pixel.

The ultrasound imaging system 100 may normalize the calculated composite score to a range from 0 to 1 and spatially map the normalized values for each unit region to generate the second combined map CM2. According to various embodiments, the normalization process may use Min-Max scaling, Z-score normalization, or a clinical data-based optimization function, and the selected method may be stored in the memory 150 to maintain the consistency of normalization.

The method for automatically determining the measurement ROI position according to one embodiment (1400) may include extracting a unit region (1404). The ultrasound imaging system 100 may extract the unit region from the second combined map based on the composite score.

For example, the ultrasound imaging system 100 may extract a single unit region with a maximum composite score from the second combined map.

As another example, the ultrasound imaging system 100 may additionally extract at least one unit region in which the average elasticity reliability for each unit region is greater than or equal to a preset threshold value. The threshold value may be adjusted by a user, and for example, the threshold value may be set to 0.7 or greater. This extraction of multiple candidate regions may provide a user with various measurement options.

The ultrasound imaging system 100 may check whether the extracted candidate regions are larger than or equal to a minimum required size (for example, 5×5 mm) and examine whether the region is maintained at a minimum distance (for example, 1 mm or more) from the image boundary. In addition, it is possible to calculate a distance from anechoic structures such as blood vessels or bile ducts, and to firstly select regions that are separated by 1 mm or more.

Morphological characteristics of a region may also be considered, and thus a region, in which a spatial distribution of the composite score that is close to circular or elliptical, may be favored. For this, various tools may be used to calculate shape complexity, and a region with lower complexity may be firstly selected.

The method for automatically determining the measurement ROI position according to one embodiment (1400) may include determining a measurement ROI position to include an extracted unit region (1405). The ultrasound imaging system 100 may determine the position of the measurement ROI to include one unit region having a maximum composite score.

The ultrasound imaging system 100 may determine a size and shape of the measurement ROI based on the estimated size of the lesion or a value set by a user through the input interface 170. For example, a circular measurement ROI with a diameter of 10 mm may be standardly used for liver fibrosis measurement, and a square measurement ROI of 20 mm may be standardly used for breast tumor evaluation.

The position of the measurement ROI may be determined based on the center of the extracted unit region, but may be fine-tuned to avoid image boundaries or artifacts. An adjustment range may be limited to within a certain number of millimeters of the original position. During the adjustment process, an optimization algorithm may be applied to maximize an average composite score within the measurement ROI.

The ultrasound imaging system 100 may additionally determine at least one measurement ROI position to include at least one unit region in which the average elasticity reliability for each unit region is greater than or equal to a threshold value. When a plurality of measurement ROIs is determined, a minimum distance between each measurement ROI may be maintained to prevent duplicate measurements. For example, the minimum distance may be set to 0.5 times a diameter of the measurement ROI.

When the ultrasound imaging system determines the position of the measurement ROI, an indicator (that is, the second indicator) for indicating the position of the measurement ROI may be displayed on the elasticity image and the elasticity reliability image, respectively. When a plurality of ROIs is determined, each position may be displayed in a different color (for example, red, blue, green) or size.

The ultrasound imaging system 100 may store information on the determined measurement ROI position in the memory 150 for reference during subsequent follow-up examinations of the same patient. Further, when a user manually adjusts the ROI position via the input interface 170, the adjusted position information may also be stored and utilized as learning data. This feedback information may be utilized to improve future automatic ROI determination algorithms.

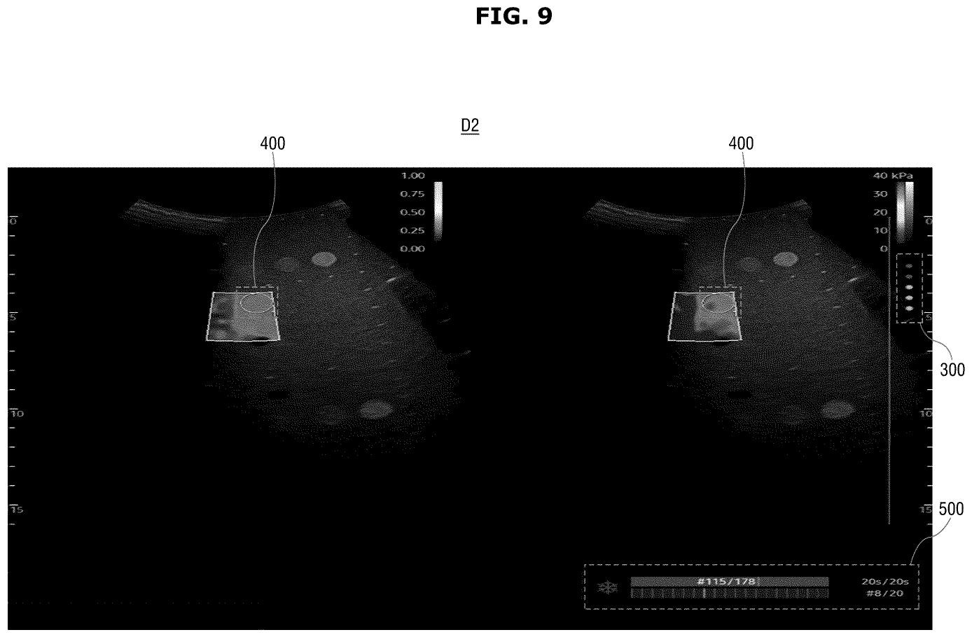

FIG. 9 is a view illustrating various indicators provided together with an image frame displayed on a display of the ultrasound imaging system according to one embodiment.

According to one embodiment, the method for controlling the ultrasound imaging system 100 may include displaying one of the extracted plurality of image frames and simultaneously displaying at least one indicator of a first indicator for indicating an elastic modulus measurement suitability of the displayed image frame, a second indicator for indicating an automatically determined measurement ROI position of the displayed image frame, and a third indicator for indicating a sort order of the displayed image frame among the extracted plurality of image frames.

Referring to FIG. 9, an image frame D2 may include the first indicator 300, the second indicator 400, and/or the third indicator 500.

In the present disclosure, an indicator may refer to any form of display means for visually conveying specific information to a user. The indicator may include graphic elements, text information, color displays, geometric shapes, animation effects, or a combination thereof. The indicator may be classified into static and dynamic display methods. The static display method may refer to displaying an indicator in a fixed position in a fixed form, while the dynamic display method may refer to changing over time or changing in response to user input. In addition, the indicator may apply various visual expression methods, such as two-dimensional flat displays, three-dimensional stereoscopic displays, translucent overlay displays, blinking displays, and gradient displays.

The indicator may include at least one display element.

The display element may refer to an individual visual element that forms the indicator. The display element may include icons, text, numbers, lines, points, surfaces, color blocks, progress bars, buttons, sliders, checkboxes, radio buttons, drop-down menus, pop-up windows, tooltips, badges, labels, symbols, shapes, graphs, charts, or a combination thereof. Each display element may have unique visual attribute, which may include color, size, shape, transparency, position, rotation, texture, border style, shadow effect, glow effect, animation effect, and the like. The display elements may be divided into active elements that allow user interaction and passive elements that only display information.

Referring to FIG. 9, the first indicator 300 may be displayed to indicate the elastic modulus measurement suitability of the displayed image frame.

In FIG. 9, the first indicator 300 is arranged in an upper right region of the image, but

this is only one embodiment. The display position of the first indicator is not limited to the drawing. According to various embodiments, the first indicator 300 may be arranged in the upper left, lower left, lower right, upper center, lower center of the image, or a separate region outside the image. In addition, a user can freely set or move the position of the first indicator through the input interface 170, and the set position information may be stored in the memory 150 and applied in the same manner in the next examination.

The first indicator 300 may be composed of a plurality of display elements, and may include at least one of a number display element, a color display element, a graph display element, a progress bar display element, or an icon display element. The first indicator 300 may determine the color, number, size, or shape of the display elements included based on a preset score range to which the elastic modulus measurement suitability belongs. For example, shades of green may be displayed as a higher suitability, and shades of red may be displayed as a lower suitability. In addition, the number or brightness of the display elements may be adjusted according to the detailed suitability even within the same score range.

Referring to FIG. 9, the second indicator 400 may be displayed to indicate the automatically determined position of measurement ROI of the displayed image frame.

The second indicator 400 may be displayed directly overlaid on the elasticity image and elasticity reliability image, thereby allowing a user to immediately identify the exact position of the measurement ROI.

As illustrated in FIG. 9, the second indicator 400 may include display elements that indicate the position of the measurement ROI in each of the two images (elasticity image and elasticity reliability image).

The second indicator 400 may be implemented as a display element of various shapes, and may include a circular display element, a rectangular display element, an oval display element, a polygonal display element, a crosshairs display element, or a combination thereof. Each display element may be implemented as a solid line, a dotted line, a dashed line, a double line, or a gradient line, and the thickness, color, and transparency of the line may be adjusted to optimize visibility. When a plurality of positions of the measurement ROI is automatically determined, each position may be displayed by being distinguished by a display element of a different color (for example, red, blue, green, and yellow), size, or shape.

Referring to FIG. 9, the third indicator 500 may be displayed to indicate the sort order of the displayed image frames among the extracted plurality of image frames.

The third indicator 500 may be arranged in the lower center region of the image in FIG. 9, but this is only one embodiment. The display position of the third indicator is not limited to the drawing. According to various embodiments, the third indicator 500 may be arranged in the upper end, left side, and right side of the image, or in a separate control panel region outside the image. In addition, the third indicator 500 may be arranged horizontally, vertically, circularly, or in a user-defined shape, and a user can set the arrangement direction and position through the input interface 170.

The third indicator 500 may be composed of a plurality of display elements, and may include at least one of a progress bar display element, a pagination display element, a tab display element, a timeline display element, a slider display element, or a number display element. The third indicator 500 may visually express the position of the current frame according to sort in order of time or sort in order of elastic modulus measurement suitability, and may provide the relative position of the current frame compared to the total number of frames, a navigation function to the previous/next frame, a function to directly move to a specific frame, and the like.

In one embodiment, the ultrasound imaging system 100 may provide interaction between the first indicator 300, the second indicator 400, and the third indicator 500. For example, when the ultrasound imaging system 100 moves to another frame via the third indicator 500, the first indicator 300 may immediately update suitability of the corresponding frame, and the second indicator 400 may newly display ROI position of the corresponding frame. In addition, when a specific suitability level is selected in the first indicator 300, a filtering function may be provided to allow only frames corresponding to the level to be displayed in the third indicator 500.

Each indicator may individually control display/hidden according to user settings, and may provide customization functions such as size adjustment, transparency adjustment, and color theme changes. This setting information may be stored in the memory 150 and managed for each user or for each diagnostic subject, and when the same user performs the next examination, previous settings may be automatically applied.

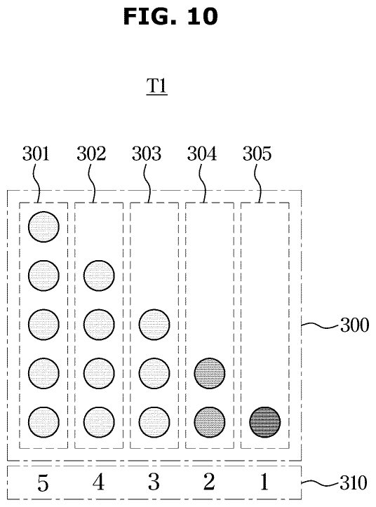

FIG. 10 is a view illustrating a configuration and display method of a first indicator according to one embodiment.

Referring to FIG. 10, a first table T1 represents a reference table for explaining the display method according to the elastic modulus measurement suitability of the first indicator 300. The first table T1 is not displayed in an actual image frame, and is an explanatory diagram that exemplarily shows various display forms of the first indicator. An elastic modulus measurement suitability display portion 310 is a portion for explaining the elastic modulus measurement suitability value indicated by each first indicator 300, and is not displayed in an actual image frame. This portion may only be used as reference information to help understanding the drawing.

The ultrasound imaging system 100 may determine the color of the first display element included in the first indicator 300 based on a preset score range to which the elastic modulus measurement suitability of the displayed image frame belongs. As can be seen in the first table T1, a region with a high suitability may be displayed in a first color (for example, shades of green), a region with an intermediate suitability may be displayed in a second color (for example, shades of yellow), and a region with a low suitability may be displayed in a third color (for example, shades of red).

In addition, the ultrasound imaging system 100 may determine the number of first

display elements to allow the number of first display elements included in the first indicator 300 to increase as the elastic modulus measurement suitability increases within the same score range. That is, the number or brightness of display elements may be adjusted according to the detailed suitability even within the same score range. As can be seen in the first table T1, a larger number of first display elements (for example, 5) may be activated and displayed in a first indicator 301 with a high suitability, and a relatively smaller number of first display elements (for example, 2 or 1) may be activated and displayed in a first indicator 305 with a low suitability.

The first display elements may be arranged in a linear arrangement, circular arrangement, or in a user-defined shape, and the size, spacing, and transparency of each display element may also be adjusted.

The ultrasound imaging system 100 may allow a user to set the display method of the first indicator through the input interface 170. For example, the color theme, shape of the display element (circle, rectangle, diamond, and the like), arrangement method, size, and the like may be adjusted according to the user's preference, and the setting information may be stored in the memory 150 and applied in the same manner to the next examination.

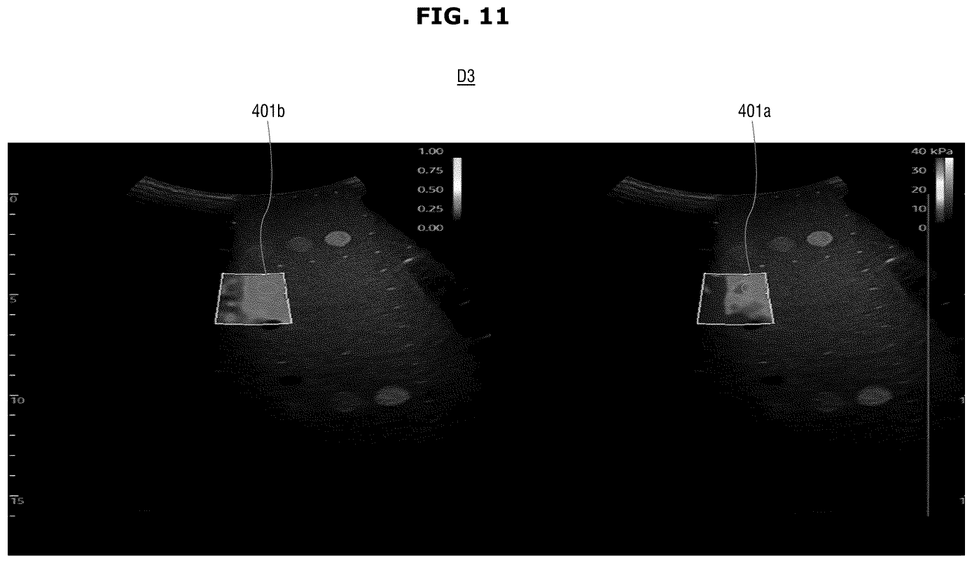

FIG. 11 is a view illustrating a configuration and display method of a second indicator according to one embodiment.

Referring to FIG. 11, an image frame D3 displayed on the display 140 may be configured in a dual view format including a first ultrasound image (for example, U1 of FIG. 3) and a second ultrasound image (for example, U2 of FIG. 3). The ultrasound imaging system 100 may display one of the extracted plurality of image frames, while simultaneously displaying a second indicator 401 for indicating the position of the measurement ROI.

The second indicator 401 may include a second display element 401a for indicating the position of the measurement ROI automatically determined in the first ultrasound image, and a third display element 401b for indicating the position of the measurement ROI automatically determined in the second ultrasound image. The second display element 401a and the third display element 401b may have the same color, the same shape, and the same size. This is to intuitively indicate that the measurement ROIs in the first ultrasound image and the second ultrasound image are set at the same corresponding anatomical positions.

The second display element 401a and the third display element 401b may be displayed in at least one of a circle, a rectangle, a cross, a diamond, or a user-defined shape. Additionally, each display element may be displayed in at least one of a solid line, a dotted line, a double line, or a line to which a gradient effect applied.

The ultrasound imaging system 100 may adjust transparency of the second indicator 401 to prevent the ultrasound image disposed thereunder from being covered. The ultrasound imaging system 100 may adjust the transparency based on user input received through the input interface 170. For example, the transparency of the second display element 401a and the third display element 401b may be determined to be a value between 30% and 80%.

The second indicator 401 may be displayed in at least one of displaying the center point, the boundary, or the entire region of the measurement ROI.

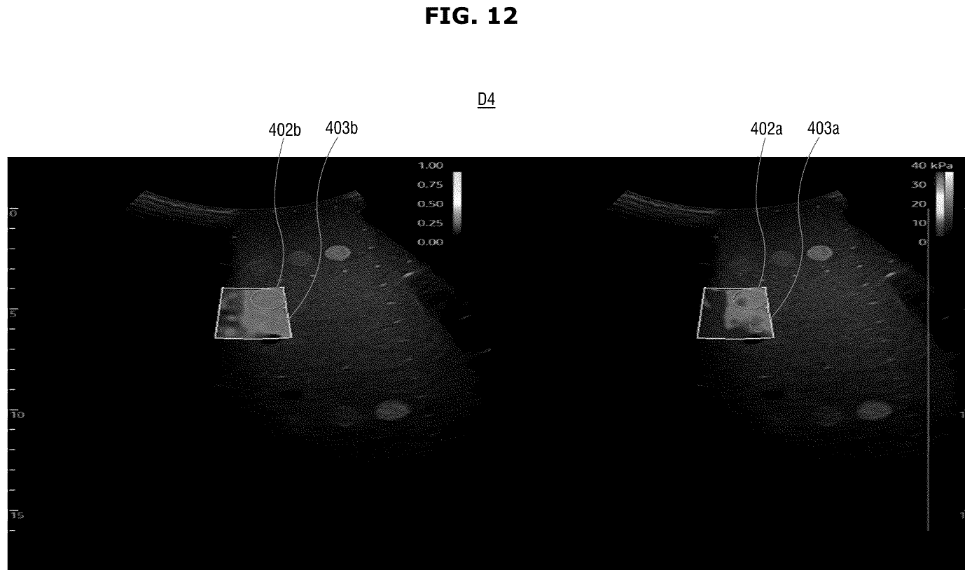

FIG. 12 is a view illustrating a configuration and display method of a plurality of second indicators according to another embodiment.

Referring to FIG. 12, an image frame D4 displayed on the display 140 is configured in a dual view format including the first ultrasound image (for example, U1 of FIG. 3) and the second ultrasound image (for example, U2 of FIG. 3), and represents a display method when a plurality of positions of measurement ROIs is set. The ultrasound imaging system 100 may simultaneously display a plurality of second indicators 402 and 403.

The second indicator 402 may indicate a position of a first measurement ROI and may include a second display element 402a displayed on the first ultrasound image, and a third display element 402b displayed on the second ultrasound image. Within the second indicator 402, the second display element 402a and the third display element 402b may have the same color, shape, and size to indicate the corresponding positions.

The second indicator 403 may indicate a position of a second measurement ROI and may include a second display element 403a displayed on the first ultrasound image and a third display element 403b displayed on the second ultrasound image. Within the second indicator 403, the second display element 403 and the third display element 403 may have the same color, shape, and size to indicate the corresponding positions.

When the plurality of second indicators 402 and 403 is displayed in a single image frame, each indicator may be distinguished by at least one visual attribute, such as a different color, shape, size, line style, or pattern. For example, the second indicator 402 may be displayed as a yellow circle, and the second indicator 403 may be displayed as a red circle. This visual differentiation may allow a user to clearly distinguish and track the plurality of measurement ROIs.

The ultrasound imaging system 100 may display measurement values or identification information corresponding to each second indicator 402 and 403. For example, at least one of the measurement ROI number, measured elastic modulus value, measurement time, or measurement depth information may be displayed in text or graphic form near each indicator.

When the plurality of second indicators is displayed, the ultrasound imaging system 100 may emphasize and display a specific second indicator based on user input received through the user interface 170. The emphasizing may be implemented by at least one of increasing the brightness of the selected indicator, emphasizing a border, or applying an animation effect. The ultrasound imaging system 100 may determine an indicator to be emphasized based on a user input received through the input interface 170.

According to various embodiments, the ultrasound imaging system 100 may allow a user to customize the display method of the second indicator 400; 401, 402, and 403 through the input interface 170. The user can set color palette, shape library, size range, transparency level (for example, 30% to 80%), line style, animation properties, and the like of the indicator, and the set values may be stored in the memory 150 and applied to subsequent examinations.

According to various embodiments, the ultrasound imaging system 100 may detect a mouse over, touch, or hovering gesture on the second indicator 400 to display additional information in the form of a pop-up, tooltip, or side panel. The additional information may include detailed measurement values of the corresponding measurement ROI, measurement parameters, measurement reliability, measurement time, or related clinical information.

According to various embodiments, the ultrasound imaging system 100 may adjust the position of the second indicator 400 using a drag-and-drop method, and the adjusted position information may be stored in the memory 150 in real time and applied to the measurement algorithm. This allows a user to manually fine-tune the automatically determined measurement ROI position. When adjusting the position, the ultrasound imaging system 100 may calculate and display the expected measurement value or measurement suitability at the adjusted position, in real time.

According to various embodiments, the ultrasound imaging system 100 may provide various display modes for the second indicator 400. For example, the display mode may be selected from an “always display” mode, a “display only when selected” mode, a “display only during measurement” mode, or an “auto-hide” mode. Each mode may be selected based on clinical workflow and user preference.

According to various embodiments, the ultrasound imaging system 100 may set the display priority of the second indicator 400. When the plurality of indicators overlaps, an indicator with a higher priority may be displayed in front or more clearly. The priority may be determined based on the size of the measurement value, measurement time, measurement reliability, or user-specified criteria.

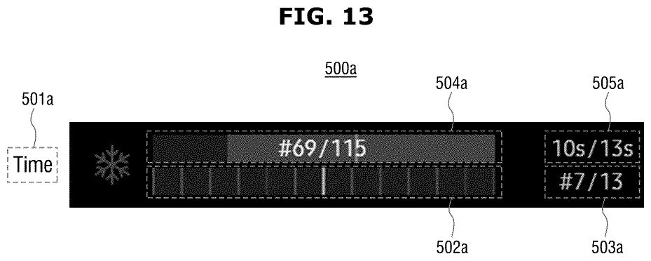

FIG. 13 is a view illustrating a configuration of the third indicator and a display method when sorted in order of time according to one embodiment.

Referring to FIG. 13, the ultrasound imaging system 100 may display a third indicator 500a for indicating the sort order of the image frames while displaying one of the extracted plurality of image frames. The third indicator 500a may intuitively indicate which position the currently displayed image frame corresponds to among the plurality of image frames sorted in order of time.

The third indicator 500a may include a fourth display element 501a that displays information regarding sort criteria. For example, as illustrated in FIG. 13, the fourth display element 501a may indicate that sort in order of time is applied, with the text “Time.” According to various embodiments, the fourth display element 501a may be displayed as a clock icon, a timeline symbol, or various other graphic elements that allow a user to intuitively recognize the concept of time.

The third indicator 500a may include a fifth display element 502a for displaying information regarding the sort order of the displayed image frames when the extracted plurality of image frames is sorted according to the sort criteria. The fifth display element 502a may be configured in the form of a color block in which a plurality of display units is arranged at equal intervals, and each display unit may correspond to one extracted image frame. A display unit corresponding to the currently displayed image frame may be highlighted and displayed. For example, the display unit corresponding to the currently displayed image frame may be emphasized in yellow or a bright color, enlarged in size, or a blinking effect may be applied thereto.

The third indicator 500a may include a sixth display element 503a indicating a sequential position of the currently displayed frames among the extracted image frames. For example, as illustrated in FIG. 13, the sixth display element 503a displayed as “#7/13” indicates that the 7^{th} frame among the 13 extracted image frames is currently being displayed. According to various embodiments, the sixth display element 503a may be modified into various formats such as “7 of 13,” “7-13,” “[7/13],” or in the form of a progress bar.

The third indicator 500a may include a seventh display element 504a indicating a position of an image frame displayed in the entire cine image. For example, as illustrated in FIG. 13, a portion displayed as “#69/115” indicates that it is the 69^{th} frame among 115 frames in the entire cine image.

The third indicator 500a may include an eighth display element 505a displaying time information. For example, as illustrated in FIG. 13, a portion displayed as “10s/13s” indicates that the current frame is displayed at 10 second point during the cine image playback, and that the total playback time is 13 seconds. According to various embodiments, the eighth display element 505a may be expressed in various ways, such as in a time code format (00:10/00:13), a percentage display (77%), or an analog clock format.

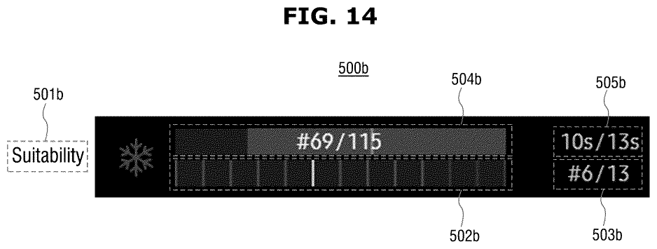

FIG. 14 is a view illustrating a configuration of a third indicator and a display method when sorted in order of elastic modulus measurement suitability according to another embodiment.

Referring to FIG. 14, the ultrasound imaging system 100 may display one of the extracted plurality of image frames and simultaneously display a third indicator 500b for indicating the sort order of the image frames. The third indicator 500b may intuitively indicate which position the currently displayed image frame corresponds to among the plurality of image frames sorted in order of elastic modulus measurement suitability.

The third indicator 500b may include a fourth display element 501b displaying information regarding the sort criteria. For example, as illustrated in FIG. 14, the fourth display element 501b may indicate that sort in order of suitability is applied, with the text “Suitability”. According to various embodiments, the fourth display element 501b may be displayed as a quality index icon, a star symbol, a checkmark, a rating badge, a reliability gauge, or other visual elements indicating measurement quality.

The third indicator 500b may include a fifth display element 502b for displaying information regarding the sort order of the image frames displayed when the extracted plurality of image frames is sorted according to the sort criteria. Similar to the fifth display element 502a of FIG. 13, the fifth display element 502b may be configured in the form of a color block in which a plurality of display units is arranged at equal intervals, and each display unit may correspond to one extracted image frame.

Particularly, in the fifth display element 502b of FIG. 14, the color of each display unit may be displayed differently according to the elastic modulus measurement suitability of the corresponding image frame. For example, a display unit corresponding to a frame with a high suitability may be displayed in green, an intermediate suitability may be displayed in yellow, and a low suitability may be displayed in red. Through this color coding, a user can grasp the suitability distribution of all extracted frames at a glance. A display unit corresponding to the currently displayed image frame may be highlighted and displayed, and for example, a border may be emphasized, a size may be enlarged, or a blinking effect may be applied.

The third indicator 500b may include a sixth display element 503b indicating a sequential position of the currently displayed frame among the extracted image frames. For example, as illustrated in FIG. 14, the sixth display element 503b displayed as “#6/13” indicates that the current frame is ranked 6^{th} among 13 extracted frames when sorted by the suitability. This may indicate a different order from the sort in order of time, and may allow a user to prioritize reviewing frames with good quality. According to various embodiments, the sixth display element 503b may be modified in various formats, such as “Rank 6 of 13,” “6th/13,” “[Suitability Rank: 6/13],” or in the form of a ranking badge.

The third indicator 500b may include a seventh indicator element 504b indicating a position of an image frame displayed in the entire cine image. For example, as illustrated in FIG. 14, a portion displayed as “#69/115” indicates that the current frame is the 69^{th} frame among 115 frames in the entire cine image. This indicates a temporal position in the original cine image, regardless of the sort in order of suitability.

The third indicator 500b may include an eighth display element 505b displaying time information. For example, as illustrated in FIG. 14, a portion displayed as “10s/13s” indicates that the current frame is displayed at 10 second point during cine image playback, and that the total playback time is 13 seconds. This allows a user to grasp the temporal context of the original image even when sorted by the suitability. According to various embodiments, the eighth display element 505b may be expressed in various ways, such as in a timestamp format (10.0 s), a frame rate display (300/390 frames), or a time bar graph format.

According to various embodiments, the ultrasound imaging system 100 may selectively display or combine components of the third indicator 500; 500a and 500b. For example, in a simple view mode, only the fourth display elements 501a and 501b and the sixth display elements 503a and 503b may be displayed to provide only key information, and in a detailed view mode, all of the fourth to eighth display elements 501 to 505 may be displayed to provide comprehensive information.

According to various embodiments, the ultrasound imaging system 100 may change the sort criteria in real time based on user input received through the input interface 170. When the sort criteria are changed, the process of rearranging the display units of the fifth display elements 502a and 502b may be displayed as a smooth transition animation, and thus a user can intuitively understand the change in the sequential position due to the change in the sort criteria.

According to various embodiments, the ultrasound imaging system 100 may support additional sort criteria to the third indicator 500. For example, the criteria may include “sort in order of measurement size,” “sort in order of reliability,” “sort in order of coefficient of variation,” “sort in order of measurement depth,” “sort in order of ROI size,” “sort in order of pressure uniformity,” or a user-defined criterion. Each sort criterion may be selected via a drop-down menu, radio buttons, toggle switches, or gesture recognition.

According to various embodiments, the fifth display elements 502a and 502b may be expressed in various visualizations other than a bar form. The fifth display elements 502a and 502b may be implemented in the form of a circular dial, a spiral timeline, a matrix grid, a thumbnail strip, a 3D cube, a radial chart, or a heat map. Each display unit may be expressed not only as a simple color block, but also as a mini thumbnail, a numeric value, an icon, a pattern, a gradient, or an animation effect.

According to various embodiments, the ultrasound imaging system 100 may include interactive function in the third indicator. When a user clicks, touches, or hovers over a specific display unit of the fifth display element 502a or 502b, detailed information about the corresponding frame may be displayed as a tooltip, and a double-click or long-press may allow immediate movement to the corresponding frame. Additionally, navigation between frames may be possible via a mouse wheel, swipe gesture, or keyboard shortcuts.

According to various embodiments, the ultrasound imaging system 100 may allow a user to customize the display position, size, transparency, color theme, and the like of the third indicator 500. The set values may be stored in the memory 150 and managed for each user profile, and optimized settings may be automatically applied depending on the examination type or clinical situation.

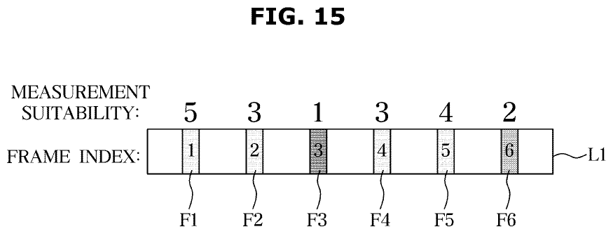

FIG. 15 is a reference view illustrating color coding scheme of a fifth display element according to one embodiment.

Referring to FIG. 15, the fifth display element 502 may include a progress bar L1 and a plurality of display units F representing a plurality of extracted image frames. For example, the fifth display element 502 may include a first display unit F1, a second display unit F2, a third display unit F3, a fourth display unit F4, a fifth display unit F5, and a sixth display unit F6. Each display unit F may be displayed in the form of a color block overlaid on the progress bar L1. The color of each color block may represent a score range to which the elastic modulus measurement suitability of the corresponding image frame belongs.

In FIG. 15, the numbers 1, 2, 3, 4, 5, and 6 displayed within each image frame F may represent frame indices, which may indicate a temporal position in which each frame is obtained within the cine image. For example, F1 is a frame is the first obtained frame, and F6 is the sixth obtained frame. These frame indices only function as unique identifiers that serve as a basis for sorting in order of time, and may not be displayed on the actual display 140.

“Measurement suitability” value displayed on the upper end of each image frame indicates an elastic modulus measurement suitability score calculated by the ultrasound imaging system 100 for the corresponding frame. For example, referring to FIG. 15, an image frame corresponding to the first display unit F1 may correspond to a suitability of 5, an image frame corresponding to the second display unit F2 may correspond to a suitability of 3, an image frame corresponding to the third display unit F3 may correspond to a suitability of 1, an image frame corresponding to the fourth display unit F4 may correspond to a suitability of 3, an image frame corresponding to the fifth display unit F5 may correspond to a suitability of 4, and an image frame corresponding to the sixth display unit F6 may correspond to a suitability of 2.

The ultrasound imaging system 100 may define a preset score range according to the elastic modulus measurement suitability and assign a specific color to the display unit F according to each range.

For example, when the elastic modulus measurement suitability of the image frame is 3 or more and 5 or less, the display unit F corresponding to the image frame may be displayed in green. Referring to FIG. 15, the first display unit F1, the second display unit F2, the fourth display unit F4, and the fifth display unit F5 may be displayed as green color blocks.

As another example, when the elastic modulus measurement suitability of the image frame is 2, the display unit F corresponding to the image frame may be displayed in yellow. Referring to FIG. 15, the sixth display unit F6 may be displayed as a yellow color block.

As another example, when the elastic modulus measurement suitability of the image frame is 1, the display unit F corresponding to the image frame may be displayed in red. Referring to FIG. 15, the third display unit F3 may be displayed as a red color block.

According to one embodiment, the ultrasound imaging system 100 may allow a user to intuitively understand the measurement quality of each frame through the color coding. The color range and threshold value may be customized by the user through the input interface 170 and automatically adjusted according to the examination type or clinical protocol.

According to one embodiment, the ultrasound imaging system 100 may arrange the plurality of display units F at equal intervals on the progress bar L1 in the fifth display element 502. When the display units are displayed too densely or overlapped, visibility for a user may be reduced, so the ultrasound imaging system 100 may arrange the display units to allow a constant interval to be maintained between each display unit.

According to one embodiment, the ultrasound imaging system 100 may dynamically adjust the interval between display units depending on the number of extracted frames. For example, when there is only one extracted frame, as in a first row of a third table T3 described below, only a single display unit may be displayed. When there are two extracted frames, as in a second row, an interval between display units may be set wide. When there are six extracted frames, as in a fifth row, an interval may be set relatively narrow.

According to one embodiment, the ultrasound imaging system 100 may display each display unit F in the form of a color block by overlaying each display unit F on the progress bar L1. The color of each display unit may be determined based on the elastic modulus measurement suitability of the corresponding frame, and a consistent color coding may be applied regardless of the sort criterion (time or suitability).

According to one embodiment, the ultrasound imaging system 100 may apply a secondary sort criterion when frames with the same suitability are present. For example, frames with the same suitability when sorted by the suitability may be further sorted by frame index (in order of time). In a forward sorting, frames with a lower frame index may be displayed first, and in a reverse sorting, frames with a higher frame index may be displayed first.

According to various embodiments, the ultrasound imaging system 100 may highlight and display a display unit corresponding to the currently displayed image frame. The highlighting may be implemented by methods such as border emphasizing, size enlargement, blinking effect, brightness increase, or arrow display.

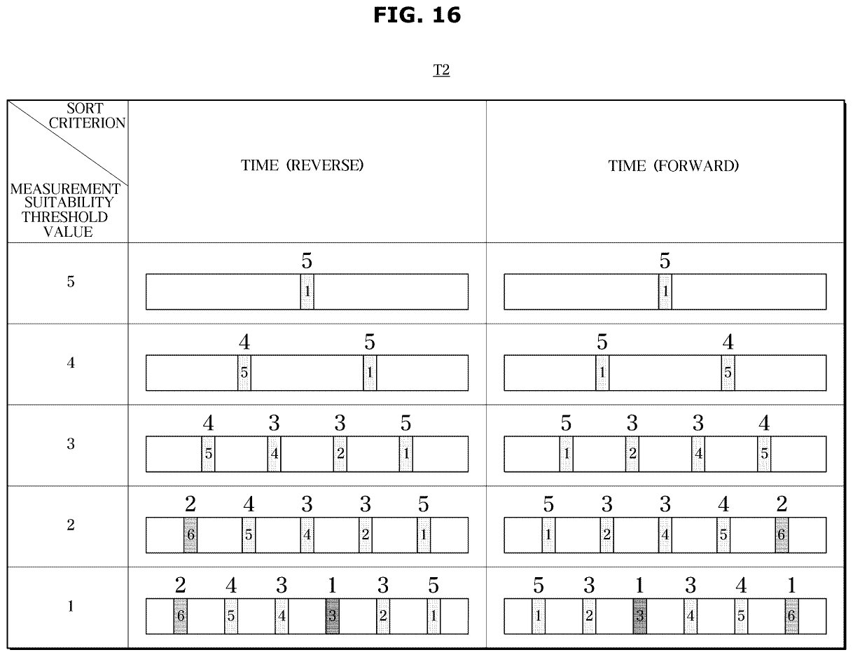

FIG. 16 is a view illustrating various implementation methods of the fifth display element when sorted in order of time according to one embodiment.

Referring to FIG. 16, a second table T2 includes various display forms of the fifth display element 502 according to a frame automatic extraction threshold value and a sorting direction when the sort criterion is time. The second table T2 is an explanatory reference table that is not displayed on an actual screen, and systematically organizes various implementation examples of the fifth display element 502.

The ultrasound imaging system 100 may automatically extract image frames having the elastic modulus measurement suitability being greater than or equal to a preset threshold value. In the second table T2 of FIG. 16, the “Measurement Suitability Threshold Value” column indicates the suitability threshold value used for image frame extraction, and the “Sorting Direction” column indicates the direction (forward or reverse) of sorting by time.

For example, when the threshold value is set to 5 and reverse sorting is applied, as in the first row and first column of the second table T2, frames having the elastic modulus measurement suitability of 5 or greater may be extracted. Referring to FIG. 15, the first display unit F1 (suitability 5) may be extracted, and only the first display unit F1 (suitability 5) may be displayed on the progress bar L1.

For example, when the threshold value is set to 5 and reverse sorting is applied, as in the first row and the second column of the second table T2, frames having the elastic modulus measurement suitability of 5 or greater may be extracted. Referring to FIG. 15, the first display unit F1 (suitability 5) may be extracted, and only the first display unit F1 (suitability 5) may be displayed on the progress bar L1.

As another example, when the threshold value is set to 4 and reverse sorting is applied, as in the second row and the first column of the second table T2, frames having the elastic modulus measurement suitability of 4 or greater may be extracted. Referring to FIG. 15, the first display unit F1 (suitability 5) and the fifth display unit F5 (suitability 4) may be extracted, and these may be arranged from left to right in the reverse frame index order (5→1). The fifth display element 502 may arrange these display units at equal intervals on the progress bar L1.

As another example, when the threshold value is set to 4 and forward sorting is applied, as in the second row and the second column of the second table T2, frames having the elastic modulus measurement suitability of 4 or greater may be extracted. Referring to FIG. 15, the first display unit F1 (suitability 5) and the fifth display unit F5 (suitability 4) may be extracted, and these may be arranged from left to right in the reverse frame index order (1→5). The fifth display element 502 may arrange these display units at equal intervals on the progress bar L1.

As another example, when the threshold value is set to 3 and reverse sorting is applied, as in the third row and the first column of the second table T2, frames having the elastic modulus measurement suitability of 3 or greater may be extracted. Referring to FIG. 15, the first display unit F1 (suitability 5), the second display unit F2 (suitability 3), the fourth display unit F4 (suitability 3), and the fifth display unit F5 (suitability 4) may be extracted, and these may be arranged from left to right in the reverse frame index order (5→4→2→1). The fifth display element 502 may arrange these display units at equal intervals on the progress bar L1.

As another example, when the threshold value is set to 3 and forward sorting is applied, as in the third row and the second column of the second table T2, frames having the elastic modulus measurement suitability of 3 or greater may be extracted. Referring to FIG. 15, the first display unit F1 (suitability 5), the second display unit F2 (suitability 3), the fourth display unit F4 (suitability 3), and the fifth display unit F5 (suitability 4) are extracted, and these may be arranged from left to right in reverse frame index order (1→2→4→5). The fifth display element 502 may arrange these display units at equal intervals on the progress bar L1.

As another example, when the threshold value is set to 2 and reverse sorting is applied, as in the fourth row and the first column of the second table T2, frames having the elastic modulus measurement suitability of 2 or greater may be extracted. Referring to FIG. 15, the first display unit F1 (suitability 5), the second display unit F2 (suitability 3), the fourth display unit F4 (suitability 3), the fifth display unit F5 (suitability 4), and the sixth display unit F6 (suitability 2) may extracted, and these may be arranged from left to right in the reverse frame index order (6→5→4→2→1). The fifth display element 502 may arrange these display units at equal intervals on the progress bar L1.

As another example, when the threshold value is set to 2 and forward sorting is applied, as in the fourth row and the second column of the second table T2, frames having the elastic modulus measurement suitability of 2 or greater may be extracted. Referring to FIG. 15, the first display unit F1 (suitability 5), the second display unit F2 (suitability 3), the fourth display unit F4 (suitability 3), the fifth display unit F5 (suitability 4), and the sixth display unit F6 (suitability 2) may be extracted, and these may be arranged from left to right in the reverse frame index order (1→2→4→5→6). The fifth display element 502 may arrange these display units at equal intervals on the progress bar L1.

As another example, when the threshold value is set to 1 and reverse sorting is applied, as in the fifth row and the first column of the second table T2, frames having the elastic modulus measurement suitability of 1 or greater may be extracted. Referring to FIG. 15, the first display unit F1 (suitability 5), the second display unit F2 (suitability 3), the third display unit F3 (suitability 1), the fourth display unit F4 (suitability 3), the fifth display unit F5 (suitability 4), and the sixth display unit F6 (suitability 2) may be extracted, and these may be arranged from left to right in the reverse frame index order (6→5→4→3→2→1). The fifth display element 502 may arrange these display units at equal intervals on the progress bar L1.

As another example, when the threshold value is set to 1 and forward sorting is applied, as in the fifth row and the second column of the second table T2, frames having the elastic modulus measurement suitability of 1 or greater may be extracted. Referring to FIG. 15, the first display unit F1 (suitability 5), the second display unit F2 (suitability 3), the third display unit F3 (suitability 1), the fourth display unit F4 (suitability 3), the fifth display unit F5 (suitability 4), and the sixth display unit F6 (suitability 2) may be extracted, and these may be arranged from left to right in the frame index order (1→2→3→4→5→6). The fifth display element 502 may arrange these display units at equal intervals on the progress bar L1.

According to various embodiments, when the number of extracted frames is small, the ultrasound imaging system 100 may improve visual clarity by setting the interval between display units wider.

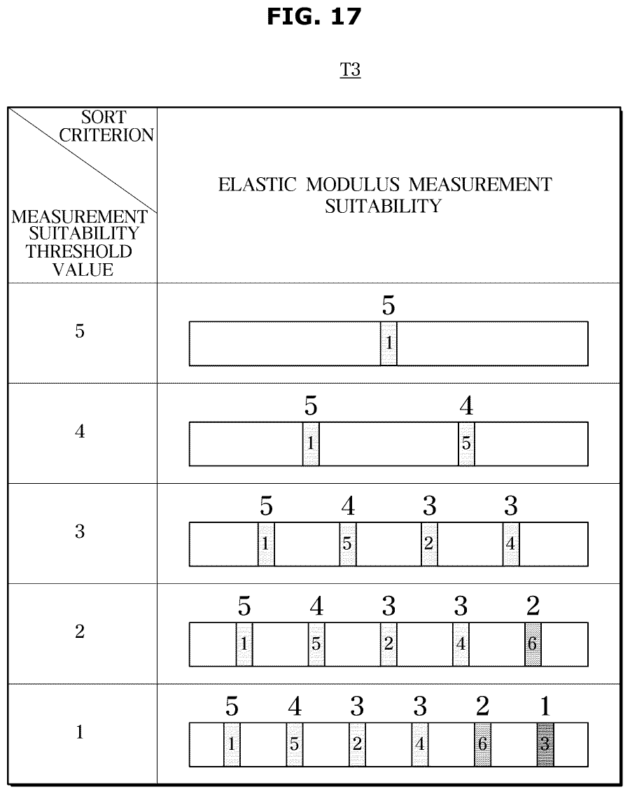

FIG. 17 is a view illustrating various implementation methods of the fifth display element when sorted in order of measurement suitability according to one embodiment.

Referring to FIG. 17, the third table T3 includes various display forms of the fifth display element 502 according to a frame automatic extraction reference value and a sorting direction when the sort criterion is the elastic modulus measurement suitability. The third table T3 is an explanatory reference table that is not displayed on the actual screen, and systematically organizes various implementation examples of the fifth display element 502.

The ultrasound imaging system 100 may sort the extracted frames in descending order according to the elastic modulus measurement suitability value. Accordingly, the ultrasound imaging system 100 may preferentially display image frames with a high elastic modulus measurement suitability. In the third table T3 of FIG. 17, the “Measurement Suitability Threshold Value” column indicates the elastic modulus measurement suitability threshold value used for the image frame extraction, and the “Sort Criterion” column indicates the elastic modulus measurement suitability.

For example, when the threshold value is set to 5, as in the first row and the first column of the third table T3, frames having the elastic modulus measurement suitability of 5 or greater may be extracted. Referring to FIG. 15, only the first display unit F1 (suitability 5) may be extracted, and only the first display unit F1 may be displayed on the progress bar L1.

As another example, when the threshold value is set to 4, as in the second row and the first column of the third table T3, frames having the elastic modulus measurement suitability of 4 or greater may be extracted. Referring to FIG. 15, the first display unit F1 (suitability 5) and the fifth display unit F5 (suitability 4) may be extracted, and these may be arranged from left to right in descending order of suitability (5→4). That is, F1 may be displayed first and F5 may be displayed later. The fifth display element 502 may arrange these display units at equal intervals on the progress bar L1.

As another example, when the threshold value is set to 3, as in the third row and the first column of the third table T3, frames having the elasticity measurement suitability of 3 or greater may be extracted. Referring to FIG. 15, the first display unit F1 (suitability 5), the second display unit F2 (suitability 3), the fourth display unit F4 (suitability 3), and the fifth display unit F5 (suitability 4) may be extracted, and these may be arranged from left to right in descending order of the elasticity measurement suitability (5→4→3(F2)→3(F4)). In the case of F2 and F4 having the same suitability, F2 may be displayed before F4 by using the chronological order as a secondary sort criterion. The fifth display element 502 may arrange these display units at equal intervals on the progress bar L1.

As another example, when the threshold value is set to 2 and reverse sorting (descending order) is applied, as in the fourth row and the first column of the third table T3, frames having the elastic modulus measurement suitability of 2 or greater may be extracted. Referring to FIG. 15, the first display unit F1 (suitability 5), the second display unit F2 (suitability 3), the fourth display unit F4 (suitability 3), the fifth display unit F5 (suitability 4), and the sixth display unit F6 (suitability 2) may be extracted, and these may be arranged from left to right in descending order of the elastic modulus measurement suitability (5→4→3(F2)→3(F4)→2). In the case of F2 and F4 having the same suitability, F2 may be displayed before F4 by using the chronological order as the secondary sort criterion. The fifth display element 502 may arrange these display units at equal intervals on the progress bar L1.

As another example, when the threshold value is set to 1 and reverse sorting (descending order) is applied, as in the fifth row and the first column of the third table T3, all frames having the elastic modulus measurement suitability of 1 or greater may be extracted. Referring to FIG. 15, the first display unit F1 (suitability 5), the second display unit F2 (suitability 3), the third display unit F3 (suitability 1), the fourth display unit F4 (suitability 3), the fifth display unit F5 (suitability 4), and the sixth display unit F6 (suitability 2) may be all extracted and these may be arranged from left to right in descending order of the elastic modulus measurement suitability (5→4→F2(3)→F4(3)→2→1). In the case of F2 and F4 having the same suitability, F2 may be displayed before F4 by using the chronological order as the secondary sort criterion. The fifth display element 502 may arrange these display units at equal intervals on the progress bar L1.

As is apparent from the above description, it is possible to provide a system configured to calculate and score an elastic modulus measurement suitability of each image frame based on elasticity image and elasticity reliability image data, thereby consistently evaluating measurement quality without subjective judgment of an examiner.

Further, it is possible to provide a system configured to automatically determine an optimal measurement ROI position by comprehensively analyzing elasticity uniformity and elasticity reliability in extracted image frames, and configured to display a plurality of ROIs without mutual interference when the plurality of ROIs is required.

Further, it is possible to provide a system configured to allow an examiner to intuitively recognize quality and position information in real time by displaying a plurality of indicators simultaneously with an image frame and providing user customization and interaction functions.

A control method of an ultrasound imaging system according to one embodiment may include obtaining a cine image, which includes a plurality of image frames, by aligning an elasticity image generated based on a shear wave tracking signal received from a probe on the

same time stamp and an elasticity reliability image generated based on parameters obtained from the shear wave tracking signal from the probe, with an ultrasound image generated based on an echo signal received from the probe; determining an elastic modulus measurement suitability of each of the plurality of image frames based on elasticity image data and elasticity reliability image data of each of the plurality of image frames; automatically extracting a plurality of image frames for measuring an elastic modulus based on the elastic modulus measurement suitability; automatically determining a position of measurement region of interest (ROI) of each of the extracted image frames based on the elasticity image data and the elasticity reliability image data of each of the extracted plurality of image frames; and displaying one of the extracted plurality of image frames and simultaneously displaying at least one of a first indicator for indicating an elastic modulus measurement suitability of the displayed image frame, a second indicator for indicating an automatically determined position of measurement ROI of the displayed image frame, and a third indicator for indicating a sort order of the displayed image frame among the extracted plurality of image frames.

The determining of the elastic modulus measurement suitability may include calculating elasticity uniformity standard deviation for each unit region from the elasticity image data of each of the plurality of image frames, and generating an elasticity uniformity STD map based on the elasticity uniformity standard deviation for each unit region; generating an average elasticity reliability map for each unit region from the elasticity reliability image data of each of the plurality of image frames; generating a first combined map for each of the plurality of image frames by synthesizing the elasticity uniformity STD map with the average elasticity reliability map; and determining the elastic modulus measurement suitability of each of the plurality of image frames by calculating an average value of the first combined map.

Additionally, an elasticity average value may be calculated from the elasticity image data and divided into the STD map to generate a representative elasticity uniformity STD map, and the representative elasticity uniformity STD map may be synthesized with the average elasticity reliability map to generate a first combined map of each of the plurality of image frames.

The displaying of one of the extracted plurality of image frames and simultaneously displaying the first indicator may include determining a color of a first display element included in the first indicator based on a preset score range to which the elastic modulus measurement suitability of the displayed image frame belongs.

The displaying of one of the extracted plurality of image frames and simultaneously displaying the first indicator may include determining the number of first display elements to allow the number of first display elements included in the first indicator to increase as the elastic modulus measurement suitability of the displayed image frame increases within the same score range.

The displaying of one of the extracted plurality of image frames may include simultaneously displaying a first ultrasound image in which the elasticity image is overlaid on the ultrasound image, and a second ultrasound image in which the elasticity reliability image is overlaid on the ultrasound image.

The displaying of one of the extracted plurality of image frames and simultaneously displaying the second indicator may include simultaneously displaying a second display element for indicating the position of the measurement ROI automatically determined on the first ultrasound image, and a third display element for indicating the position of the measurement ROI automatically determined on the second ultrasound image.

The automatically determining of the position of the measurement ROI of each of the extracted image frames may include generating an elasticity uniformity map by calculating an elasticity uniformity for each unit region from the elasticity image data of each of the extracted image frames; generating an average elasticity reliability map for each unit region from the elasticity reliability image data of each of the extracted image frames; calculating a composite score for each unit region by applying a first weighted value to the elasticity uniformity map and by applying a second weighted value to the average elasticity reliability, map and generating a second combined map on which the composite score for each unit region is reflected; extracting a unit region based on the composite score from the second combined map; and determining a position of the measurement ROI to allow the measurement ROI to include the extracted unit region.

The generating of the second combined map may include determining the first and second weighted values based on a user identification number obtained through a user interface or a communication interface.

The extracting of the unit region based on the composite score from the second combined map may include extracting one unit region in which the composite score is a maximum value. The determining of the position of the measurement ROI to allow the measurement ROI to include the extracted unit region may include determining a position of the measurement ROI so as to include one unit region in which the composite score is a maximum value.

The extracting of the unit region based on the composite score from the second combined map may include additionally extracting at least one unit region in which the average elasticity reliability for each unit region is greater than or equal to a preset threshold value. The determining of the position of the measurement ROI to allow the measurement ROI to include the extracted unit region may include additionally determining at least one position of the measurement ROI so as to include at least one unit region in which the average elasticity reliability for each unit region is greater than or equal to the preset threshold value.

The displaying of one of the extracted plurality of image frames and simultaneously displaying the second indicator may include displaying, based on the position of the measurement ROIs being automatically determined as a plurality of positions, each position with a plurality of second indicators of different colors.

The displaying of one of the extracted plurality of image frames and simultaneously displaying the third indicator may include displaying a fourth display element provided to display information about a sort criterion; and displaying a fifth display element for displaying information about the sort order of the displayed image frames in response to the extracted plurality of image frames being sorted by the sort criterion.

The sort criterion may correspond to any one of sort in order of time or sort in order of elastic modulus measurement suitability. The control method of the ultrasound imaging system may include determining the sort criterion based on user input.

The displaying of the fifth display element may include overlaying and displaying a plurality of display units corresponding to each of the extracted plurality of image frames at equal intervals according to the sort criterion, on a sort order display element; and highlighting and displaying a display unit corresponding to the displayed image frame.

The displaying of the fifth display element may include displaying each of the plurality of display units corresponding to each of the extracted plurality of image frames in a color determined based on a preset score range to which the elastic modulus measurement suitability of the corresponding extracted image frame belongs.

The automatically extracting of the plurality of image frames for measuring the elastic modulus based on the elastic modulus measurement suitability may include automatically extracting the image frame as an image frame for measuring the elastic modulus based on the elastic modulus measurement suitability of the image frame being greater than or equal to a reference value; and displaying, through a user interface, that none of the plurality of image frames has been extracted for measuring the elastic modulus based on the elastic modulus measurement suitability of all of the plurality of image frames included in the cine image being less than the reference value.

The automatically extracting of the plurality of image frames for measuring the elastic modulus based on the elastic modulus measurement suitability may include providing a guide for inducing resetting of the elastic modulus measurement suitability threshold value through the user interface based on the elastic modulus measurement suitability of all of the plurality of image frames included in the cine image being less than the reference value.

An ultrasound imaging system according to one embodiment may include: a probe configured to receive an echo signal and a shear wave tracking signal; an image processor configured to generate an ultrasound image based the echo signal, configured to generate an elasticity image based on the shear wave tracking signal, configured to generate an elasticity reliability image based on parameters obtained from the shear wave tracking signal, and configured to generate a cine image, which includes a plurality of image frames, by aligning the ultrasound image, the elasticity image and the elasticity reliability image; a display displaying the plurality of image frames; and a processor configured to determine an elastic modulus measurement suitability of each of the plurality of image frames based on elasticity image data and elasticity reliability image data of each of the plurality of image frames; configured to automatically extract a plurality of image frames for measuring an elastic modulus based on the elastic modulus measurement suitability; configured to automatically determine a position of measurement region of interest (ROI) of each of the extracted image frames based on the elasticity image data and the elasticity reliability image data of each of the extracted plurality of image frames; and configured to control the display to display one of the extracted plurality of image frames and simultaneously display at least one of a first indicator for indicating an elastic modulus measurement suitability of the displayed image frame, a second indicator for indicating an automatically determined position of measurement ROI of the displayed image frame, and a third indicator for indicating a sort order of the displayed image frame among the extracted plurality of image frames.

The processor may be configured to calculate elasticity uniformity standard deviation for each unit region from the elasticity image data of each of the plurality of image frames, and generate an elasticity uniformity STD map based on the elasticity uniformity standard deviation for each unit region; configured to generate an average elasticity reliability map for each unit region from the elasticity reliability image data of each of the plurality of image frames; configured to generate a first combined map for each of the plurality of image frames by synthesizing the elasticity uniformity STD map with the average elasticity reliability map; and configured to determine the elastic modulus measurement suitability of each of the plurality of image frames by calculating an average value of the first combined map.

Additionally, an elasticity average value may be calculated from the elasticity image data and divided into the STD map to generate a representative elasticity uniformity STD map, and the representative elasticity uniformity STD map may be synthesized with the average elasticity reliability map to generate a first combined map of each of the plurality of image frames.

The processor may be configured to determine a color of a first display element included in the first indicator based on a preset score range to which the elastic modulus measurement suitability of the displayed image frame belongs.

The processor may be configured to determine the number of first display elements to allow the number of first display elements included in the first indicator to increase as the elastic modulus measurement suitability of the displayed image frame increases within the same score range.

As is apparent from the above description, a process of calculating suitability and extracting region of interest (ROI) may be automated, and thus it is possible to perform measurement with consistent quality regardless of skill of an examiner, and to significantly improve standardization and reproducibility of diagnostic results.

Further, only high-quality frames may be automatically selected and low-reliability regions may be immediately identified, thereby reducing unnecessary re-measurement and shortening an examination time, and increasing diagnostic accuracy by reducing false positives and false negatives.

Further, indicator-based visualization and customization functions may allow a user to intuitively check the anatomical structure, elasticity properties, and reliability information of a target region for diagnosis on a single screen, thereby simplifying clinical workflow and improving decision-making speed.

Meanwhile, the disclosed embodiments may be embodied in the form of a recording medium storing instructions executable by a computer. The instructions may be stored in the form of program code and, when executed by a processor, may generate a program module to perform the operations of the disclosed embodiments. The recording medium may be embodied as a computer-readable recording medium.

The computer-readable recording medium includes all kinds of recording media in which instructions which can be decoded by a computer are stored. For example, there may be a Read Only Memory (ROM), a Random Access Memory (RAM), a magnetic tape, a magnetic disk, a flash memory, and an optical data storage device.

Storage medium readable by machine, may be provided in the form of a non-transitory storage medium. “Non-transitory storage medium” means that the storage medium is a tangible device and does not contain a signal (e.g., electromagnetic wave), and this term includes a case in which data is semi-permanently stored in a storage medium and a case in which data is temporarily stored in a storage medium. For example, “non-transitory storage medium” may include a buffer in which data is temporarily stored.

The method according to the various disclosed embodiments may be provided by being included in a computer program product. Computer program products may be traded between sellers and buyers as commodities. Computer program products are distributed in the form of a device-readable storage medium (e.g., compact disc read only memory (CD-ROM)), or are distributed directly or online (e.g., downloaded or uploaded) between two user devices (e.g., smartphones) through an application store (e.g., Play Store™). In the case of online distribution, at least a portion of the computer program product (e.g., downloadable app) may be temporarily stored or created temporarily in a device-readable storage medium such as the manufacturer's server, the application store's server, or the relay server's memory.

Additional aspects of the disclosure will be set forth in part in the description which follows and, in part, will be obvious from the description, or may be learned by practice of the disclosure.

While the present disclosure has been particularly described with reference to exemplary embodiments, it should be understood by those of skilled in the art that various changes in form and details may be made without departing from the spirit and scope of the present disclosure.

1. A control method of an ultrasound imaging system comprising:
obtaining a cine image, which comprises a plurality of image frames, by aligning an elasticity image generated based on a shear wave tracking signal received from a probe on the same time stamp and an elasticity reliability image generated based on parameters obtained from the shear wave tracking signal from the probe, with an ultrasound image generated based on an echo signal received from the probe;
determining an elastic modulus measurement suitability of each of the plurality of image frames based on elasticity image data and elasticity reliability image data of each of the plurality of image frames;
automatically extracting a plurality of image frames for measuring an elastic modulus based on the elastic modulus measurement suitability;
automatically determining a position of measurement region of interest (ROI) of each of the extracted image frames based on the elasticity image data and the elasticity reliability image data of each of the extracted plurality of image frames; and
displaying one of the extracted plurality of image frames and simultaneously displaying at least one of a first indicator for indicating an elastic modulus measurement suitability of the displayed image frame, a second indicator for indicating an automatically determined position of measurement ROI of the displayed image frame, and a third indicator for indicating a sort order of the displayed image frame among the extracted plurality of image frames.

2. The control method of the ultrasound imaging system of claim 1, wherein
the determining of the elastic modulus measurement suitability comprises:
calculating elasticity uniformity standard deviation for each unit region from the elasticity image data of each of the plurality of image frames, and generating an elasticity uniformity STD map based on the elasticity uniformity standard deviation for each unit region;
generating an average elasticity reliability map for each unit region from the elasticity reliability image data of each of the plurality of image frames;
generating a first combined map for each of the plurality of image frames by synthesizing the elasticity uniformity STD map with the average elasticity reliability map; and
determining the elastic modulus measurement suitability of each of the plurality of image frames by calculating an average value of the first combined map.

3. The control method of the ultrasound imaging system of claim 1, wherein
the displaying of one of the extracted plurality of image frames and simultaneously displaying the first indicator comprises:
determining a color of a first display element included in the first indicator based on a preset score range to which the elastic modulus measurement suitability of the displayed image frame belongs.

4. The control method of the ultrasound imaging system of claim 3, wherein
the displaying of one of the extracted plurality of image frames and simultaneously displaying the first indicator comprises:
determining the number of first display elements to allow the number of first display elements included in the first indicator to increase as the elastic modulus measurement suitability of the displayed image frame increases within the same score range.

5. The control method of the ultrasound imaging system of claim 1, wherein
the displaying of one of the extracted plurality of image frames comprises:
simultaneously displaying a first ultrasound image in which the elasticity image is overlaid on the ultrasound image, and a second ultrasound image in which the elasticity reliability image is overlaid on the ultrasound image.

6. The control method of the ultrasound imaging system of claim 5, wherein
the displaying of one of the extracted plurality of image frames and simultaneously displaying the second indicator comprises:
simultaneously displaying a second display element for indicating the position of the measurement ROI automatically determined on the first ultrasound image, and a third display element for indicating the position of the measurement ROI automatically determined on the second ultrasound image.

7. The control method of the ultrasound imaging system of claim 1, wherein
the automatically determining of the position of the measurement ROI of each of the extracted image frames comprises:
generating an elasticity uniformity map by calculating an elasticity uniformity for each unit region from the elasticity image data of each of the extracted image frames;
generating an average elasticity reliability map for each unit region from the elasticity reliability image data of each of the extracted image frames;
calculating a composite score for each unit region by applying a first weighted value to the elasticity uniformity map and by applying a second weighted value to the average elasticity reliability map, and generating a second combined map on which the composite score for each unit region is reflected;
extracting a unit region based on the composite score from the second combined map; and
determining a position of the measurement ROI to allow the measurement ROI to include the extracted unit region.

8. The control method of the ultrasound imaging system of claim 7, wherein
the generating of the second combined map comprises:
determining the first and second weighted values based on a user identification number obtained through a user interface or a communication interface.

9. The control method of the ultrasound imaging system of claim 7, wherein
the extracting of the unit region based on the composite score from the second combined map comprises:
extracting one unit region in which the composite score is a maximum value,
wherein the determining of the position of the measurement ROI to allow the measurement ROI to include the extracted unit region comprises:
determining a position of the measurement ROI so as to include one unit region in which the composite score is a maximum value.

10. The control method of the ultrasound imaging system of claim 9, wherein
the extracting of the unit region based on the composite score from the second combined map comprises:
additionally extracting at least one unit region in which the average elasticity reliability for each unit region is greater than or equal to a preset threshold value,
wherein the determining of the position of the measurement ROI to allow the measurement ROI to include the unit region comprises:
additionally determining at least one position of the measurement ROI so as to include at least one unit region in which the average elasticity reliability for each unit region is greater than or equal to the preset threshold value.

11. The control method of the ultrasound imaging system of claim 10, wherein
the displaying of one of the extracted plurality of image frames and simultaneously displaying the second indicator comprises:
displaying, based on the position of the measurement ROIs being automatically determined as a plurality of positions, each position with a plurality of second indicators of different colors.

12. The control method of the ultrasound imaging system of claim 1, wherein
the displaying of one of the extracted plurality of image frames and simultaneously displaying the third indicator comprises:
displaying a fourth display element provided to display information about a sort criterion; and
displaying a fifth display element for displaying information about the sort order of the displayed image frames in response to the extracted plurality of image frames being sorted by the sort criterion.

13. The control method of the ultrasound imaging system of claim 12, wherein
the sort criterion corresponds to any one of sort in order of time or sort in order of elastic modulus measurement suitability,
wherein the control method of the ultrasound imaging system comprises:
determining the sort criterion based on user input.

14. The control method of the ultrasound imaging system of claim 12, wherein
the displaying of the fifth display element comprises:
overlaying and displaying a plurality of display units corresponding to each of the plurality of image frames extracted according to the sort criterion at equal intervals on a sort order display element; and
highlighting and displaying a display unit corresponding to the displayed image frame.

15. The control method of the ultrasound imaging system of claim 14, wherein
the displaying of the fifth display element comprises:
displaying each of the plurality of display units corresponding to each of the extracted plurality of image frames in a color determined based on a preset score range to which the elastic modulus measurement suitability of the corresponding extracted image frame belongs.

16. The control method of the ultrasound imaging system of claim 1, wherein
the automatically extracting of the plurality of image frames for measuring the elastic modulus based on the elastic modulus measurement suitability comprises:
automatically extracting the image frame as an image frame for measuring the elastic modulus based on the elastic modulus measurement suitability of the image frame being greater than or equal to a reference value; and
displaying, through a user interface, that none of the plurality of image frames has been extracted for measuring the elastic modulus based on the elastic modulus measurement suitability of all of the plurality of image frames included in the cine image being less than the reference value.

17. The control method of the ultrasound imaging system of claim 15, wherein
the automatically extracting of the plurality of image frames for measuring the elastic modulus based on the elastic modulus measurement suitability comprises:
providing a guide for inducing resetting of the elastic modulus measurement suitability threshold value through the user interface based on the elastic modulus measurement suitability of all of the plurality of image frames included in the cine image being less than the reference value.

18. An ultrasound imaging system comprising:
a probe configured to receive an echo signal and a shear wave tracking signal;
an image processor configured to generate an ultrasound image based the echo signal, configured to generate an elasticity image based on the shear wave tracking signal, configured to generate an elasticity reliability image based on parameters obtained from the shear wave tracking signal, and configured to generate a cine image, which comprises a plurality of image frames, by aligning the ultrasound image, the elasticity image and the elasticity reliability image;
a display displaying the plurality of image frames; and
a processor configured to determine an elastic modulus measurement suitability of each of the plurality of image frames based on elasticity image data and elasticity reliability image data of each of the plurality of image frames;
configured to automatically extract a plurality of image frames for measuring an elastic modulus based on the elastic modulus measurement suitability;
configured to automatically determine a position of measurement region of interest (ROI) of each of the extracted image frames based on the elasticity image data and the elasticity reliability image data of each of the extracted plurality of image frames; and
configured to control the display to display one of the extracted plurality of image frames and simultaneously display at least one of a first indicator for indicating an elastic modulus measurement suitability of the displayed image frame, a second indicator for indicating an automatically determined position of measurement ROI of the displayed image frame, and a third indicator for indicating a sort order of the displayed image frame among the extracted plurality of image frames.

19. The ultrasound imaging system of claim 18, wherein
the processor is configured to:
calculate elasticity uniformity standard deviation for each unit region from the elasticity image data of each of the plurality of image frames, and generate an elasticity uniformity STD map based on the elasticity uniformity standard deviation for each unit region;
generate an average elasticity reliability map for each unit region from the elasticity reliability image data of each of the plurality of image frames;
generate a first combined map for each of the plurality of image frames by synthesizing the elasticity uniformity STD map with the average elasticity reliability map; and
determine the elastic modulus measurement suitability of each of the plurality of image frames by calculating an average value of the first combined map.

20. The ultrasound imaging system of claim 18, wherein
the processor is configured to determine a color of a first display element included in the first indicator based on a preset score range to which the elastic modulus measurement suitability of the displayed image frame belongs.
